(19) United States
(12) Patent Application Publication
(10) Pub. No.: US 2013/0072946 A1
(43) Pub. Date: Mar. 21, 2013

(54) LIGATION DEVICE, CLIP UNIT, CLIP MANIPULATION DEVICE, AND ENDOSCOPE SYSTEM

(71) Applicant: Kazuhiro TERADA, Kanagawa (JP)

(72) Inventor: Kazuhiro TERADA, Kanagawa (JP)

(73) Assignee: FUJIFILM Corporation, Tokyo (JP)

(21) Appl. No.: 13/598,425

(22) Filed: Aug. 29, 2012

(30) Foreign Application Priority Data
Sep. 15, 2011 (JP) 2011-202025

(51) Int. Cl. A61B 17/128; A61B 17/122

(52) U.S. Cl. 606/142; 606/157

(57) ABSTRACT

A clip unit used in a ligation device for ligating a biological tissue, includes: arms protruding from a rear end of the clip unit to define a space for accommodating a hook; and engagement claw parts respectively extending from rear ends of the arms toward a front side, in which: when the clip unit and a transmission member are connected, front ends of the engagement claw parts abut against a rear side inclined surface of the hook.

### CROSS-REFERENCE TO RELATED APPLICATIONS

This application is based on and claims priority under 35 USC 119 from Japanese Patent Application No. 2011-202025 filed on Sep. 15, 2011; the entire content of which is incorporated herein by reference.

### BACKGROUND

1. Technical Field

The invention relates to a ligation device, a clip unit, a clip manipulation device, and an endoscope system.

2. Related Art

In general, a ligation device for an endoscope includes a clip unit configured to ligate a biological tissue and located at a front end of an elongated flexible sheath extending from a handle manipulation unit. The clip unit ligates a biological tissue in a desired direction by reciprocating a manipulation wire in the axial direction of the sheath or by rotating the manipulation wire around the axis of the sheath at the handle manipulation unit, in which the manipulation wire is inserted through the sheath. Finally, the clip unit is detained in a body cavity in the state where it grasps the biological tissue by strongly pulling the manipulation wire to the handle manipulation side.

In this type of ligation device, the connection of the clip unit and the hook is manually performed. Therefore, it is desired that such a ligation device is configured in such a manner that the connection process can be performed as simply as possible. Ligation devices, in which the connection process is simplified, are proposed in, for example, Patent Document 1 (JP-A-2002-191609) and Patent Document 2 (JP-A-2005-58627).

The ligation device disclosed in Patent Document 1 includes a clip, a fastening ring fitted on the clip, a connection member inserted into the fastening ring and engaged with the clip, and a manipulation member having a hook part engaged with the connection member to close the clip in the fastening ring. With this ligation device, the clip and the manipulation member are connected by engaging the hook part with the connection member, and the connection member is adapted to be fractured by pulling the manipulation member to allow the connection of the clip and the manipulation member to be released.

In addition, the ligation device disclosed in Patent Document 2 has a connection loop which is formed from a wire rod having a spring characteristic and attached to an area in the vicinity of the rear end of a clip in a state where the rear end portion of the connection loop is crossed in an X shape and opened, thereby forming an X-shaped opening. With the ligation device of this construction, the connection or connection release of the connection loop and a pin may be conducted by introducing the pin into or pulling the pin out from the connection loop at the X-shaped opening of the rear end of the connection loop.

### SUMMARY OF THE INVENTION

However, the ligation device disclosed in Patent Document 1 may leave unnecessary fragments in the device after the connection release, and then the fractured pieces remain, it is impossible to load the next clip. For that reason, there is a problem in that it is required to remove the unnecessary fragments at each time of ligation action, thereby increasing the length of time for maneuver.

The ligation device disclosed in Patent Document 2 is configured such that the manipulation wire and the clip are connected without a connection member. As such, the problem of fragments of Patent Document 1 may be solved. However, there is a problem in that mounting the wire and clip is difficult to perform per se, and a resultant force at the time of connection release is largely variable by a fine error of the curved shape of the connection loop.

Accordingly, an object of the present invention is to provide a ligation device in which a manipulation wire and a clip unit are directly connected, and which is configured to allow the clip connection process to be readily performed as well as to suppress the variation of the resultant force for release at the time of connection release, the clip unit, the clip manipulation device, and an endoscope system.

(1) According to an aspect of the invention, a ligation device for ligating a biological tissue, includes:

a flexible elongated sheath member;

a manipulation unit arranged in a base end side of the sheath member;

a transmission member inserted into the sheath member to be capable of being reciprocated, and configured to transmit a driving force from the manipulation unit; and

a clip unit detachably attached to a front end of the transmission member opposite to the manipulation unit in a longitudinal axis of the sheath member;

in which the transmission member includes a hook fixed on a front end of the transmission member to be engaged with the clip unit, and

the hook includes a front side inclined surface and a rear side inclined surface formed in order from a front end side of the hook to a rear side thereof,

a sectional area of the front side inclined surface perpendicular to a central axis of the hook being gradually increased, and

a sectional area of the rear side inclined surface perpendicular to the central axis being gradually reduced,

in which the clip unit includes arms protruding from the rear end of the clip unit to define a space for accommodating the hook, and engagement claw parts respectively extending from rear ends of the arms toward a front side, and in which, when the clip unit and the transmission member are connected, front ends of the engagement claw parts abut against the rear side inclined surface of the hook.

(2) A clip unit used in the ligation device of (1), includes:

the arm; and

the engagement claw parts.

(3) A clip manipulation device of the ligation device of (1), includes:

the sheath member;

the manipulation unit; and

the transmission member.

(4) An endoscope system includes:

the ligation device of (1); and

an endoscope including a treatment instrument insertion channel, through which the sheath member is inserted.

With the inventive ligation device, clip unit, clip manipulation device and endoscope system, it is easy to perform a clip connection process and is possible to suppress unevenness in the resultant force for release at the time of connection release while configuring the manipulation wire and the clip unit to be directly connected.

### BRIEF DESCRIPTION OF THE DRAWINGS

FIG. 1 illustrates an entire configuration of a ligation device for describing an exemplary embodiment of the present invention.

FIG. 2 illustrates the configuration of a clip unit partially in cut-away.

FIG. 3 is an exploded perspective view of the clip unit illustrated in FIG. 2.

FIGS. 4A and 4B are a side view and a front view of the clip body, respectively.

FIG. 5 is a bottom view of the clip body seen from the direction indicated by arrow V1 in FIG. 4B.

FIGS. 6A and 6B are a side view and a front view of the front end side of an inner fastening ring, respectively, and FIG. 6C is the side view of the rear end side of the inner fastening ring.

FIG. 7 is a cross-sectional view taken along line A-A in FIG. 6B.

FIG. 8A is a perspective view illustrating a state in which the clip body and the inner fastening ring are engaged with each other without showing an outer fastening ring, and FIG. 8B is a cross-sectional view taken along line B-B in FIG. 8A.

FIGS. 9A and 9B are a side view of the front end side of the outer fastening ring, and a front view thereof, respectively.

FIG. 10 is a cross-sectional view taken along line C-C in FIG. 9A.

FIG. 11 is a cross-sectional view illustrating the outer fastening ring and the inner fastening ring in the state where they are engaged with each other.

FIG. 12 is a cross-sectional view of the handle manipulation unit of the ligation device.

FIG. 13 is an enlarged cross-sectional view of the front end part of the manipulation unit body and an area in the vicinity of the front end part of the slitting section illustrated in FIG. 12.

FIG. 14 is an exploded perspective view of a manipulation wire anchor for anchoring the manipulation wire to a slider.

FIG. 15 is a sectioned perspective view of the slider body.

FIG. 16 is a cross-sectional view of a front end sheath.

FIG. 17 is a front view of the hook and the guide bead which are fixed to the front end of the manipulation wire.

FIG. 18 is a cross-sectional view taken along line D-D in FIG. 17.

FIG. 19 is a perspective view illustrating, partially in cut-away, the clip body and the hook in the state where they are engaged with each other.

FIG. 20 is a perspective view of a clip case.

FIG. 21 is an exploded perspective view of the clip case.

FIG. 22 is a side view of the clip case.

FIGS. 23A to 23D) are explanatory views illustrating a sequence of mounting the clip unit of the clip case to the hook.

FIG. 24 is an enlarged view of the part indicated by arrow A3 in the bottom case of FIG. 23A.

FIG. 25 is a perspective view of the part indicated by arrow A3 in the bottom case of FIG. 23A.

FIGS. 26A to 26C are explanatory views illustrating the hook from the state where the hook is inserted into the connection tail section of the clip body to the state where the hook is connected to the connection tail section in a step-by-step manner.

FIG. 27 is an enlarged explanatory view illustrating the flap parts in the state of being closed;

FIG. 28A is an explanatory view illustrating a state where the clip body and the outer fastening ring are connected with each other, and FIG. 28B is an explanatory view illustrating the front end sheath curved in the connected state of FIG. 28A.

FIG. 29 is an explanatory view illustrating a configuration example in which the guide bead is replaced by a guide spring.

FIG. 30 is an explanatory view illustrating a configuration example in which a guide bead is arranged at the rear end of the guide spring.

FIGS. 31A to 31F are explanatory views illustrating states where the arm parts of the clip unit are expanded from the front end sheath.

FIGS. 32A to 32E are explanatory views illustrating the actions of the arm parts of the clip unit, from expanding after having protruded to the outside from the front end sheath to ligating a biological tissue, in a step-by-step manner.

FIG. 33A is an explanatory view illustrating the appearance of an expansion retaining protrusion of the inner fastening ring entering into the slit part in the base end section, and FIG. 33B is an explanatory view illustrating the appearance of the expansion retaining protrusion abutting against the front end of the slit part.

FIG. 34 is an explanatory view illustrating a rotation operation of the manipulation unit body in relation to the finger pull ring.

FIG. 35 is an explanatory view illustrating a disengagement prevention protrusion of an arm part locked by a stepped part formed by a constricted spot of the outer fastening ring.

FIG. 36 is a view shown in the direction indicated by arrow V2 in FIG. 35.

FIG. 37 is an explanatory view illustrating, partially in cut-away, the clip unit in the state where the clip unit has completed the ligation.

FIGS. 38A and 38B are explanatory views illustrating the hook and the connection tail section in the state where the hook and the connection tail section are engaged with each other, and in the state where the hook and the connection tail section is started to be disconnected, respectively.

FIGS. 39A to 39C are explanatory views illustrating a process of releasing the J-shaped claw parts from the engagement in a step-by-step manner.

FIG. 40A is a partial configuration view of a connection tail section formed with a pair of engagement claw parts, and FIG. 40B is a bottom view of FIG. 40A.

FIG. 41A is a partial configuration view of a connection tail section with an engagement claw part arranged in an opening formed in an arm part, and FIG. 41B is a bottom view of FIG. 41A.

FIG. 42 is an explanatory view illustrating a construction in which a reinforcement rib is provided to extend in the longitudinal direction of each of the arm parts with a bending point of the arm part as the center.

FIG. 43 is a cross-sectional view taken along line E-E of FIG. 42.

FIG. 44 is an explanatory view illustrating acting forces loaded to an abutting point in a state where the under-head taper part and the front ends of the J-shaped claw parts start to abut against one another in the hook part of the front end of the hook.

FIG. 45 is an explanatory view schematically illustrating a resultant force vector W.

FIGS. 46A and 46B are explanatory views illustrating a state in which the inclination angle of the under-head taper part is set to be larger than ψ₀, and a state in which the inclination angle of the under-head taper part is set to be smaller than ψ₀, respectively.

FIG. 47 is an explanatory view illustrating the abutting direction of a J-shaped claw part which abuts against an abutting point of the under-head taper part.

### DETAILED DESCRIPTION OF EMBODIMENTS OF THE INVENTION

Now, exemplary embodiments of the present invention will be described with reference to drawings.

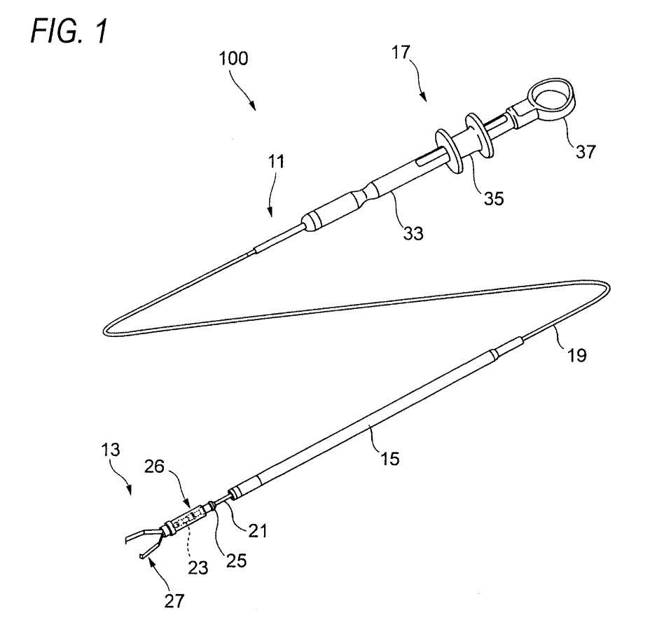

FIG. 1 is an entire configuration view illustrating a ligation device as a medical treatment instrument for describing an exemplary embodiment of the present invention.

A ligation device 100 includes a clip manipulation device 11 which is loaded with a clip unit 13. The clip manipulation device 11 includes a front end sheath 15 configured as an insertion part to be inserted into a channel of an endoscope (not shown) to be inserted into a body cavity, and a handle manipulation unit 17 located at the base end side of the front end sheath 15. The front end sheath 15 and the handle manipulation unit 17 are interconnected by a base end sheath 19, and a manipulation wire 21 is inserted through the inside of the front end sheath 15 and the inside of the base end sheath 19 to be capable of being reciprocated in the axial direction.

The front end sheath 15 and the base end sheath 19 are configured as a guide tube formed by, for example, a densely wound stainless steel coil. The manipulation wire 21 is formed by a stranded metal wire with a proper elasticity, such as, for example, a stainless steel and a NiTi alloy. In addition, to the front end of the manipulation wire 21 which is opposite to the handle manipulation unit 17, a hook 23 and a guide bead 25 are fixed in this order from the front end.

The clip unit 13 includes a fastening ring 26 formed as a cylindrical body, and a clip body 27 supported in the fastening ring 26, and the fastening ring 26 is mounted on the hook 23 provided at the front end of the manipulation wire 21.

The handle manipulation unit 17 includes: a manipulation unit body 33 to which the base end side of the base end sheath 19 is fixed to be rotatable about the axis thereof; a slider connected to be restrained from being rotated in relation to the manipulation wire 21; and a finger pull ring 37 provided at the rear end of the manipulation unit body 33 to be rotatable. The slider 35 is arranged to be restrained from being rotated in relation to the manipulation unit body 33 but to be capable of reciprocating in the axial direction of the sheath.

The handle manipulation unit 17 is capable of reciprocating the clip unit 13 connected to the manipulation wire 21 at the front end of the front end sheath 15 in the axial direction of the sheath by relatively moving the manipulation unit body 33 and the slider 35 in a longitudinal direction. In addition, by rotating the manipulation unit body 33 and the slider around the axis thereof, the clip unit 13 connected to the manipulation wire 21 is allowed to be rotated about the sheath axis.

That is, the clip manipulation device 11 is configured such that, when the slider 35 is moved away from the finger pull ring 37, the manipulation wire 21 is moved in the direction for making the manipulation wire 21 protrude forward from the front end sheath 15, and to the contrary, when the slider 35 is moved toward the finger pull ring 37, the manipulation wire 21 is moved in the direction for making the manipulation wire 21 be pulled into the front end sheath 15. In addition, when the manipulation unit body 33 and the slider 35 are rotated around the axis, the manipulation wire 21 is rotated together with the slider 35. The clip unit 13 loaded in the front end side of the manipulation wire 21 is maintained in the state in which it is restrained from being rotated by the hook 23, which will be described later. Accordingly, the clip unit 13 is released by the extension of the manipulation wire 21, and rotationally driven by the rotation of the manipulation wire 21.

The manipulation wire 21 is capable of using an appropriate wire which may readily transmit the rotation of one end thereof to the other end. For example, even a torque wire may be used which is difficult to produce rollover. In addition, a cable obtained by wire-drawing a stainless steel may be also used.

In the following description, a direction directed toward the finger pull ring 37 of the handle manipulation unit 17 of the ligation device 100 is referred to as a base end direction or rear side, and a direction directed toward the front end of the clip body 27 is referred to as a distal end direction or front side. In addition, the axial direction of the fastening ring 26 of the clip unit 13, as well as the axial direction of the front end sheath 15, the base end sheath 19, and the manipulation wire 21 will be referred to as a longitudinal axis direction.

<Clip Unit>

Next, the configuration of the clip unit 13 will be described in detail.

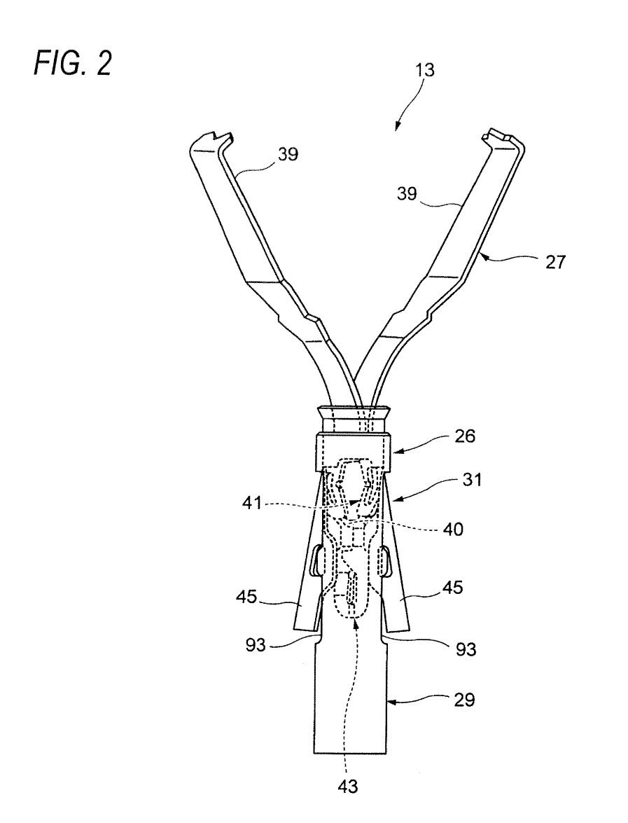

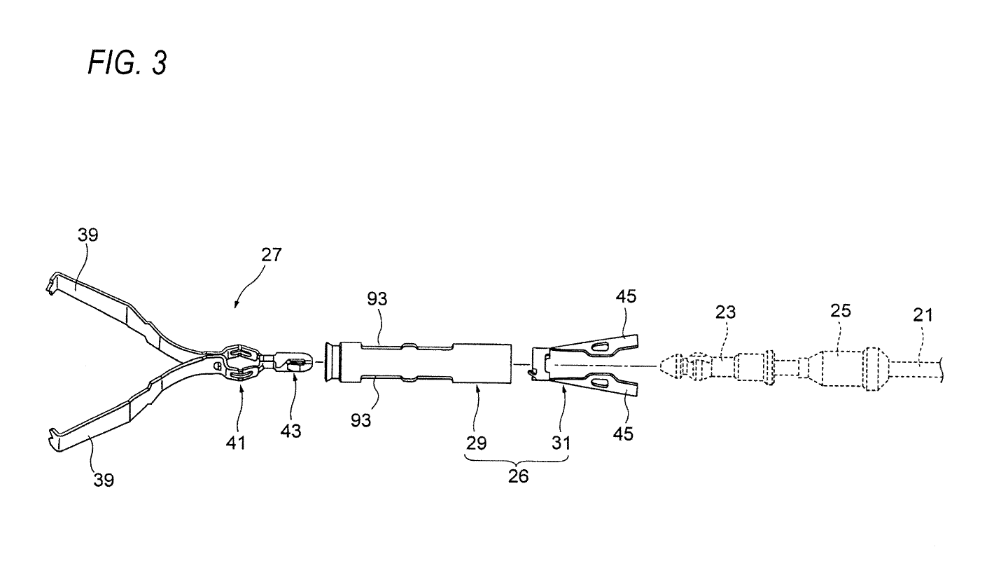

FIG. 2 is a configuration view illustrating, partially in cut-way, the clip unit, and FIG. 3 is an exploded perspective view of the clip unit. The clip unit 13 includes a fastening ring 26 and a clip body 27. The fastening ring 26 is constituted with an outer fastening ring 29 formed as a cylindrical body, and an inner fastening ring 31 inserted into the inside of the cylindrical body of the outer fastening ring 29.

The clip body 27 includes: a pair of arm parts 39, 39 which are expanded and biased in relation to each other; a base end section 41 formed in a loop shape for interconnecting the base ends of the arm parts 39, 39; a connection tail section 43 formed on the base end section 41 at the side opposite to the pair of arm parts 39, 39 (at the rear side). The arm parts 39, 39 may be closed to each other when inserted into the fastening ring 26, so that a biological tissue can be grasped by the arm parts 39, 39.

All the clip body 27, the outer fastening ring 29, and the inner fastening ring 31 may be formed from a metallic material, such as a stainless steel. The clip body 27 is formed by bending a band-shaped resilient metallic plate material in a U-shape. The outer fastening ring 29 is a cylindrical member and accommodates the inner fastening ring 31 within the inside thereof. The inner fastening ring 31 includes a pair of flap parts 45, 45 which are configured to be expanded and biased diametrically outward by elastic resilience.

The outer fastening ring 29 is formed with a pair of flap protruding holes 93, 93 at positions corresponding to the flap parts 45, 45 of the inner fastening ring 31, so that when the inner fastening ring 31 is accommodated in the outer fastening ring 29, the flap parts 45, 45 partially protrude radially outward from the flap protruding holes 93, 93.

The clip unit 13 is formed by inserting the base end section 41 and the connection tail section 43 of the clip body 27 into the outer fastening ring 29, which accommodates the inner fastening ring 31, from the front side of the outer fastening ring 29. A hook 23 is engaged with the clip unit 13, in which the hook 23 is provided at the front end of the manipulation wire 21 and inserted into the clip unit 13 from the rear side of the clip unit 13.

<Clip Body>

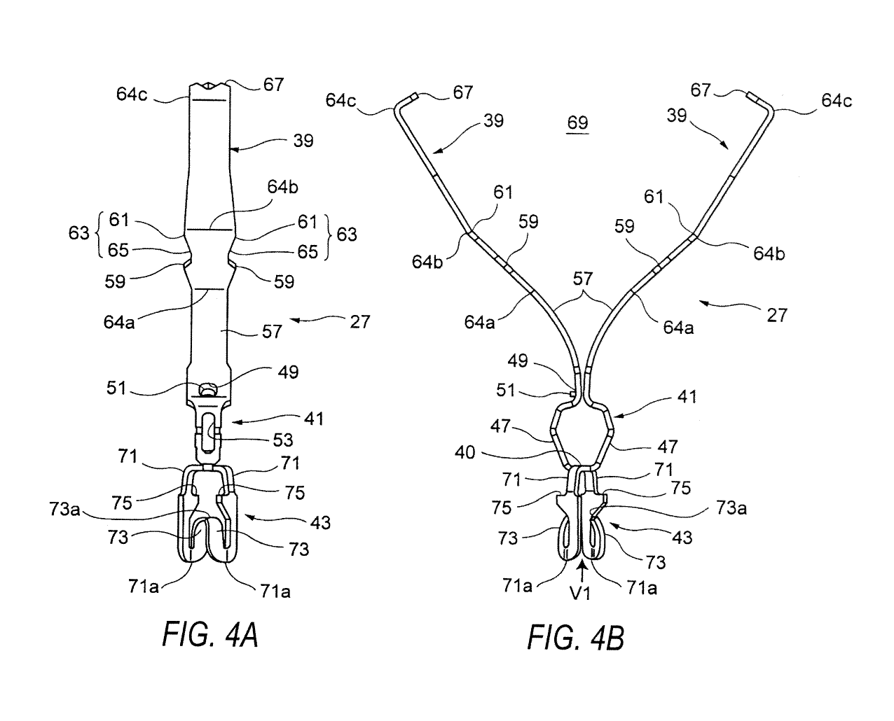

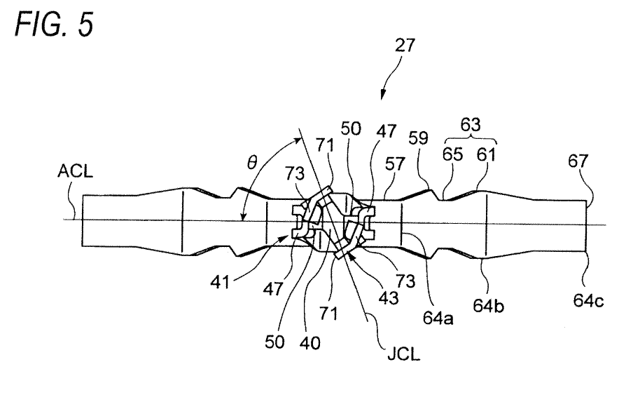

FIGS. 4A and 4B are a side view and a front view of the clip body, respectively, and FIG. 5 is a bottom view of the clip body viewed in the direction indicated by arrow V1 in FIG. 4B.

The clip body 27 is formed by bending a band-shaped metallic plate material in such a manner that a pair of the arm parts 39, 39, the base end section 41 and the connection tail section 43 are integrally formed. The base end section 41 and the connection tail section 43 extend from the connection base section 40 which has a flat surface which is perpendicular to the longitudinal central axis of the clip body 27. A pair of loop parts 47, 47 extend from the connection base section 40 and abut against each other at the arm part 39 side of the loop parts 47, 47 (loop ends), whereby the base end section 41 is formed in a loop shape in the entirety. The loop parts 47, 47 are arranged symmetrically, and connected to the arm parts 39, 39, respectively, in the side opposite to the connection base section 40.

At the abutting loop ends, one of the loop parts 47, 47 is formed with an engagement hole 49, and the other of the loop parts 47, 47 is formed with a deviation prevention claw 51 for engaging with the engagement hole 49. As the engagement hole 49 and the deviation prevention claw 51 are engaged with each other, the pair of arm parts 39, 39 are prevented from being deviated in the longitudinal axis direction and in the direction perpendicular to the longitudinal axis direction. In addition, each of the loop parts 47, 47 is formed with a slit part 53 along the loop.

The arm parts 39, 39, which are provided to extend further beyond the loop end of the loop parts 47, 47, are formed by band-shaped resilient metallic plate material members which are symmetrically arranged to be opposed to each other. The arm parts 39, 39 include: linear plate parts 57 with a predetermined width in the direction perpendicular to the expanding direction of the arm parts 39, 39; disengagement prevention protrusions 59 which are wider than the linear plate parts 57; middle parts 63, each of which is formed with a pull-in prevention protrusion 61 via a taper part 65; and front end parts 67 configured to be capable of grasping a biological tissue therebetween, in this order from the base end section 41. The arm parts 39, 39 are biased to be expanded from each other with reference to the engagement hole 49 and the deviation prevention claw 51 by the elastic resilience of the clip body 27.

A pair of the arm parts 39, 39 are formed in circular arc shapes with a curvature in such a manner that the linear plate parts 57 are bulging inwardly each other. At the front end sides of the linear plate parts 57, first bending points 64a are provided where the arm parts 39, 39 are bent outward, and in the vicinity of the pull-in prevention protrusions 61 of the middle parts 63, second bending points 64b are provided where the arm parts 39, 39 are bent inwardly. In addition, the arm parts 39, 39 are inwardly bent at third bending points 64c positioned closer to the front end side than the middle parts 63, thereby forming the front end parts 67.

When a pair of the arm parts 39 are opened by the elastic resilience of the clip body 27, the pair of front end parts 67, 67 are largely spaced from each other, thereby forming a grasping gap 69 between the front end parts 67, 67. When the arm parts 39 are closed, the front end parts 67, 67 approach each other to be capable of grasping a biological tissue. In addition, as illustrated in FIG. 4, each of the front ends of the front end parts 67, 67 may be formed to have an obtuse-angled convex part and an obtuse-angled concave part to be engaged with each other, or may have a linear shape.

The connection tail section 43 includes: a pair of plate-shaped arms 71 extending from the connection base section 40 of the base end section 41 in the direction opposite to the pair of arm parts 39; and J-shaped claw parts 73 as engagement claw parts, which are U-turned at the rear ends 71a of the arms 71, respectively, and extend from the rear ends 71a of the arms 71 toward the front side (in the direction to the arm parts 39). The pair of arms 71 define a space for accommodating the hook 23. Each of the J-shaped claw parts 73 is inclined inwardly at the front end 73a in advance, in which the J-shaped claw parts 73 are inclined more inwardly than the arms 71. In addition, each of the arms 71 extending from the connection base section 40 with a predetermined width has a wider part at the central area thereof, and engagement step parts 75 are formed by the wider parts.

As illustrated in FIG. 5, the connection tail section 43 is formed in such a manner that an arm part center line ACL and a connection tail section center line JCL are different in phase by an angle θ (for example, 40 degrees to 70 degrees, preferably 55 degrees to 60 degrees), wherein the arm part center line ACL extends through the center of the pair of arm parts 39 which are provided to extend from the base end section 41, and the connection tail section center line JCL extends through the center of a pair of arms 71 of the connection tail section 43 which are provided to extend from the connection base section 40 of the base end section 41. That is, the expansion direction of the arm parts 39 are determined to intersect the expansion direction of the connection tail section 43 with the angle θ.

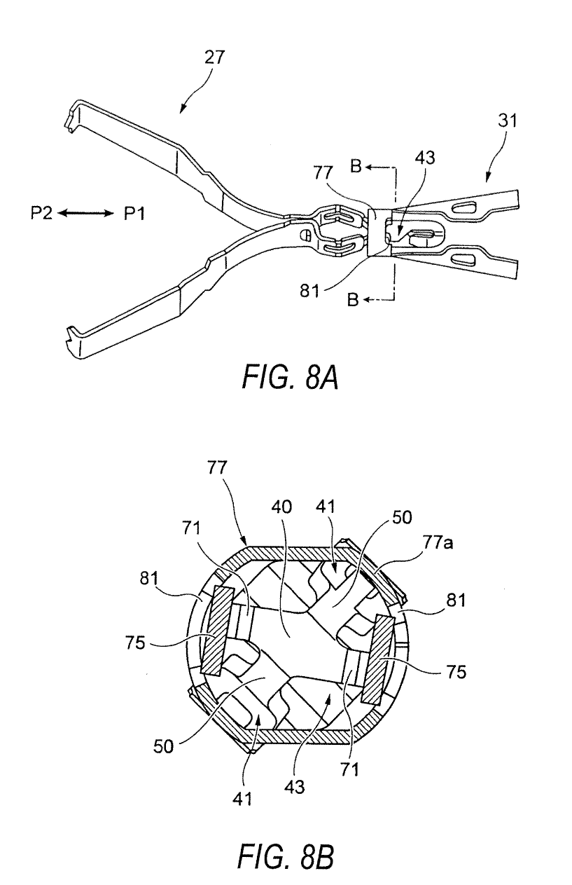

In addition, the loop parts 47, 47 of the base end section 41 are connected to the side surfaces of the flat part of the connection base section 40 via constricted spots 50 illustrated in FIG. 5 (see FIG. 8B), respectively. As a pair of the loop parts 47, 47 are connected to the connection base section 40 via the constricted spots 50, external forces applied to the arm parts 39, 39 are retrieved at the constricted spots 50, and deformation is prevented from reaching the connection base section 40 and the connection tail section 43. The constricted spots 50 also prevent external forces from reaching the arm parts 39, 39 from the connection tail section 43.

<Inner Fastening Ring>

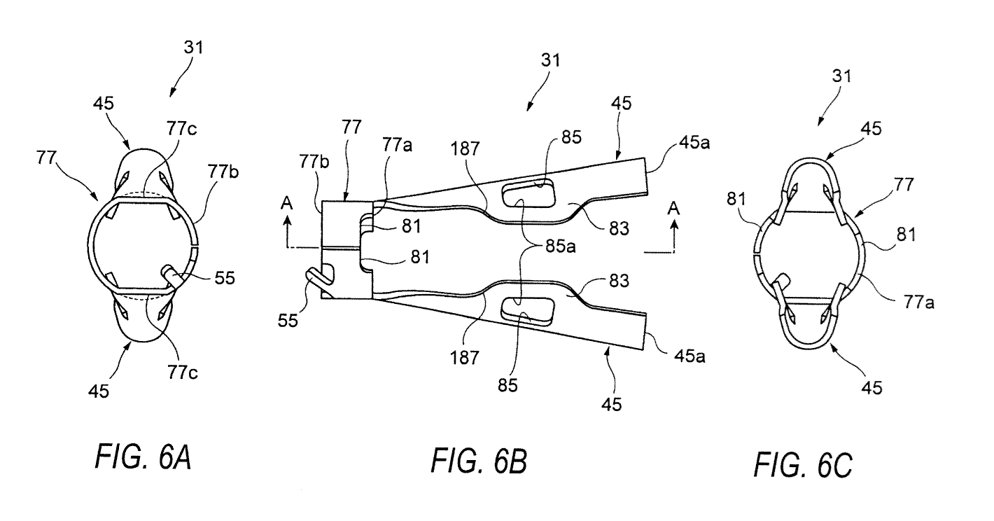

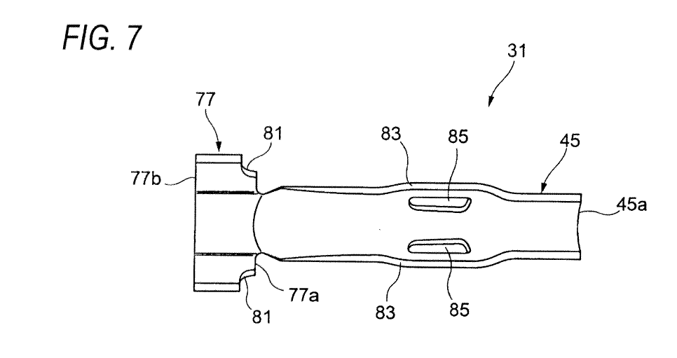

FIGS. 6A and 6B are a side view and a front view of the front end side of the inner fastening ring, respectively, and FIG. 6C is a side view of the rear end side of the inner fastening ring. FIG. 7 illustrates a cross-sectional view taken along line A-A in FIG. 6B.

The inner fastening ring 31 includes a ring part 77, and a pair of flap parts 45, 45 extending from a one side end face 77a. The pair of flap parts 45, 45 are formed by slightly expanding free ends 45a from the axial direction at two diametrically outer edge positions on the one side end face 77a of the ring part 77.

On the one side end face 77a to which the flap parts 45, 45 are connected, a pair of recesses 81, 81 are formed, and the engagement step parts 75 of the clip body 27 (see FIG. 4) are engaged in the recesses 81, 81, respectively. In addition, on the front end face 77b of the ring part 77 opposite to the one side end face 77a, an expansion retaining protrusion 55 is formed to protrude from the ring part 77 to an inclined front side facing diametrically inward. The expansion retaining protrusion 55 will be described later.

Each of the flap parts 45, 45 is formed to have a circular arc shape in a diametrical cross-section perpendicular to the axial direction, and is formed with a bulge part 83 opposite to the other flap part 45 at the axially central area thereof, in which the length of the circular arc in the diametrical cross-section is increased at the bulge part 83. The bulge parts 83 are formed with flap locking holes 85, 85, respectively. The inner fastening ring 31 is formed in such a manner that when the flap parts 45 are closed, the outer diameter of the inner fastening ring 31 is substantially equal to or slightly smaller than the inner diameter of the outer fastening ring 29.

The inner fastening ring 31 and the clip body 27 are engaged with each other as follows. FIG. 8A is a state in which the clip body 27 and the inner fastening ring 31 are engaged with each other without showing an outer fastening ring 29, and FIG. 83 is a cross-sectional view taken along line B-B in FIG. 8A. As shown in FIG. 8A, the connection tail section 43 of the clip body 27 is inserted into the ring part 77 of the inner fastening ring 31 in the P1 direction in FIG. 8A. At this time, as illustrated in FIG. 8B, the engagement step parts 75 of the connection tail section 43 are snugly fitted in the recesses 81 provided in the one side end face 77a of the ring part 77, such that the engagement step parts 75 and the recesses 81 are engaged with each other. Accordingly, even when an attempt is made to pull out the clip body 27 from the inner fastening ring 31 in the P2 direction in FIG. 8A, the engagement step parts 75 and the recesses 81 abut against each other such that they cannot be separated from each other.

<Outer Fastening Ring>

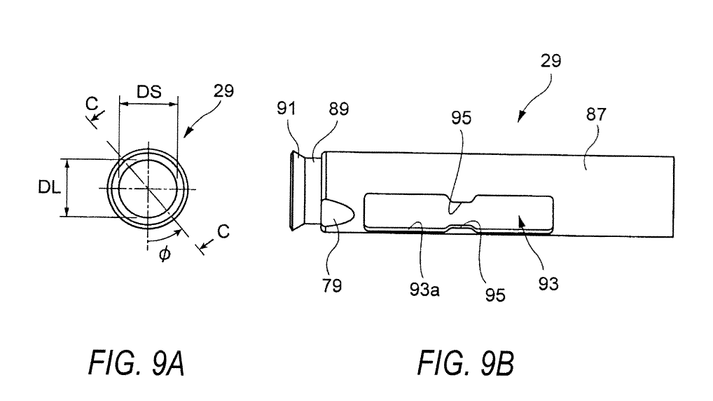

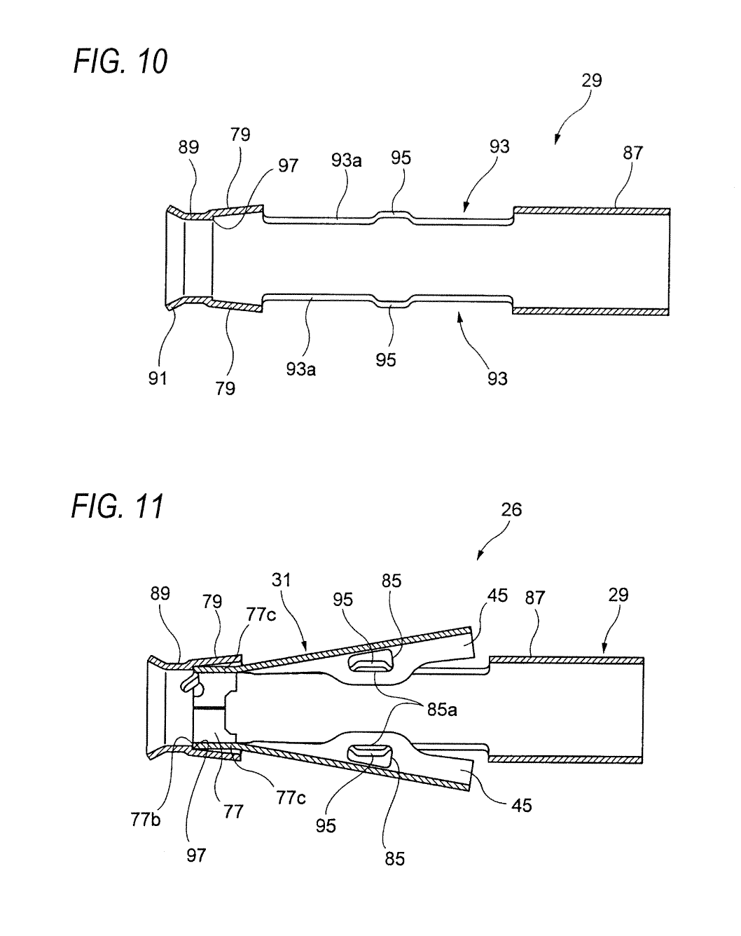

FIGS. 9A and 9B are a side view at the front end side and a front view of the outer fastening ring, respectively, and FIG. 10 is a cross-sectional view taken along line C-C in FIG. 9A.

The outer fastening ring 29 includes a cylindrical section 87, a constricted spot 89 formed by reducing the diameter of the cylindrical section 87 in the vicinity of the front end of the cylindrical section 87, and a taper section 91 extending from the constricted spot 89 in such a manner that the diameter of the taper section 91 is gradually increased as approaching the front end side. The inner diameter of the cylindrical section 87 is slightly larger than the outer diameter of the ring part 77 of the inner fastening ring 31 (see FIG. 6), such that the inner fastening ring 31 is capable of being fitted in the inside of the outer fastening ring 29. In addition, in the cylindrical section 87, a pair of flap protruding holes 93 are formed to be elongated along the axis, wherein the flap protruding holes 93 are arranged opposite to each other. Each longer side 39a forming the periphery of each of the flap protruding holes 93 is formed with flap opening prevention taps 95, 95 substantially at the central areas thereof in such a manner that the flap opening prevention taps 95, 95 protrude toward one another in the circumferential direction of the cylindrical section 87.

As illustrated in FIG. 9A, the inner face of the constricted spot 89 of the outer fastening ring 29 is formed in a substantially oval shape, so that the diameter of the major axis DL and the diameter of minor axis DS are formed in different sizes. Line C-C in FIG. 9A is a line that interconnects the centers of the widths of the flap protruding holes 93, 93 in the circumferential direction, wherein line C-C indicates a central position in the circumferential direction where the flap protruding holes 93, 93 are formed. The line C-C has a slope of a predetermined angle (for example, about 45°), and the flap protruding holes 93, 93 are formed to correspond the phase angle of the connection tail section 43 of the clip body 27.

As illustrated in FIG. 10, an inner diameter step part 97 is formed over the entire circumference of the boundary of the inner peripheries of the cylindrical section 87 and the constricted spot 89. Between the cylindrical section 87 and the constricted spot 89, a pair of planar parts 79 are formed at circumferential positions substantially the same with the flap protruding holes 93, wherein the planar parts 79 gradually reduce the diameter from the outer diameter of the cylindrical section 87 to an outer diameter in the vicinity of the outer diameter of the constricted spot 89 along the axis.

For this reason, the diametrical cross-sectional shape of the area of the planar part 79 between the cylindrical section 87 and the constricted spot 89 has parallel parts formed by the planar parts 79 and circular arc parts formed by the cylindrical section 87. This diametrical cross-sectional shape is similar to the diametrical cross-sectional shape of the ring part 77 (see FIG. 6) of the inner fastening ring 31, and the inner diameter of the outer fastening ring 29 is slightly larger than the outer diameter of the ring part 77 of the inner fastening ring 31.

FIG. 11 is a cross-sectional view illustrating the outer fastening ring 29 and the inner fastening ring 31 in a state where they are engaged with each other.

The outer fastening ring 29 and the inner fastening ring 31 are engaged with each other by inserting the inner fastening ring 31 into the cylindrical section 87 of the outer fastening ring 29 in such a manner that the ring part 77 side is positioned at the forefront. The inner fastening ring 31 is positioned in the axial direction as the front end face 77b of the ring part 77 abuts against the inner diameter step part 97 of the constricted spot 89. In addition, the position of the inner fastening ring 31 in the rotating direction is determined by fitting the planar parts 77c of the ring part 77 of the inner fastening ring 31 in the inner periphery of the parts formed with the planar parts 79 in the outer fastening ring 29, and fitting the ring part 77 of the inner fastening ring 31 in the outer fastening ring 29.

In addition, the flap opening prevention taps 95 of the outer fastening ring 29 are respectively inserted into the flap locking holes 85 provided in the pair of flap parts 45 of the inner fastening ring 31, and the flap opening prevention taps 95 respectively abut against one side 85a of the flap locking holes 85. As a result, the flap parts 45 configured to be expanded by elastic resilience are prevented from being excessively opened by abutting against the flap opening prevention taps 95, and the expansion angle of the flap parts 45 can be securely maintained at a predetermined angle established at the time of design.

In addition, since the maximum outer diameter of the loop parts 47 of the clip body 27 is determined in a size that makes the loop parts 47 abut against the taper section 91 of the outer fastening ring 29, the inner fastening ring 31 and the clip body 27, which are formed integrally with each other, are prevented from being deviated to the rear side in relation to the outer fastening ring 29. Furthermore, the clip body 27 is inseparably engaged with the inner fastening ring 31. For this reason, the clip body 27 and the outer fastening ring 29 will not be deviated from each other by vibration at the time of transportation or the like.

<Handle Manipulation Unit>

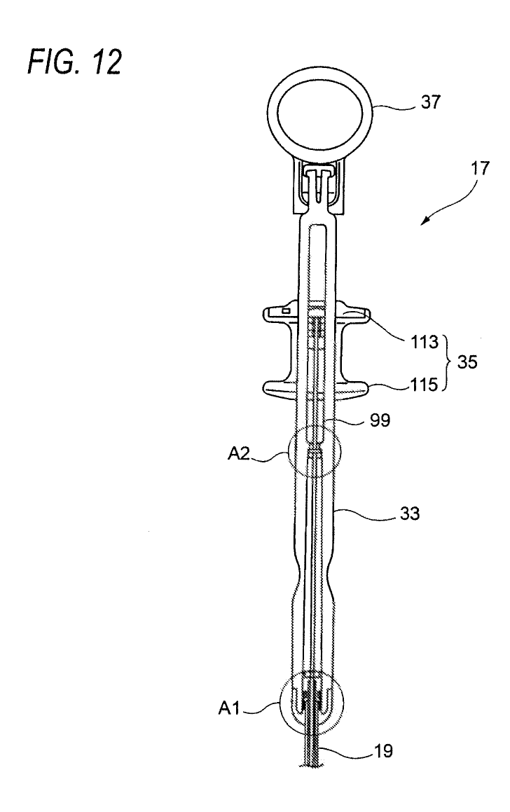

FIG. 12 is a cross-sectional view of the handle manipulation unit of the ligation device.

As described above, the handle manipulation unit 17 includes a manipulation unit body 33 to which the base end side of the base end sheath 19 is fixed to be rotatable around the axis, a slider 35, and a finger pull ring 37. The manipulation unit body 33 is provided with a slitting section 99 formed with a slit along the axial direction, and the slider 35 is configured to be movable in the axial direction within the slit forming extension. The slider consists of a slider body 115 and a manipulation wire anchor 113.

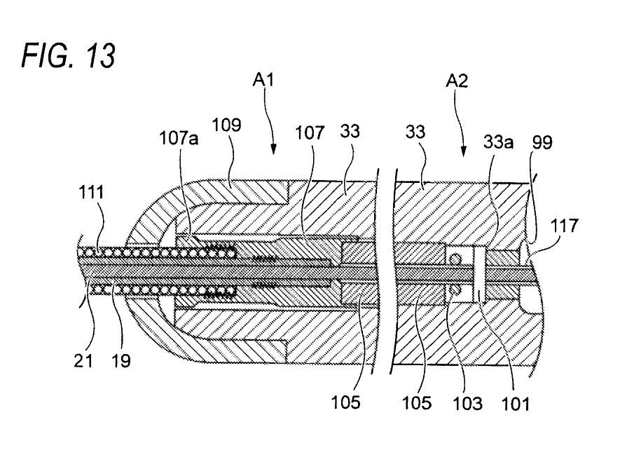

FIG. 13 is an enlarged cross-sectional view of the front end part A1 of the manipulation unit body 33 and a part A2 in the vicinity of the front end part of the slitting section 99 illustrated in FIG. 12.

A fixing clasp 107 is provided at the front end of the manipulation unit body 33, wherein a base end sheath 19 with the manipulation wire 21 inserted therein, and an bending prevention spring 111 configured to cover and protect the base end sheath 19 are inserted into the front end part 107a of the fixing clasp 107 along the axis. On the front end of the manipulation unit body 33, a cap 109 is press-fitted.

The manipulation wire 21, which extends out from the base end sheath 19, extends to the slider 35 from the base end of the fixing clasp 107. The manipulation wire 21 is inserted which is covered by the buckling prevention pipe 117 through the guide pipe 105 arranged in the base end side of the fixing clasp 107. The guide pipe 105 extends to an area in the vicinity of the front end part of the slitting section 99 to slidably accommodate the buckling prevention pipe 117. In addition, the buckling prevention pipe 117 extends to the slider 35 to retain the manipulation wire 21 in the linear shape.

The front end of the buckling prevention pipe 117 is slidably fitted in the guide pipe 105, and when the slider 35 is slid, specifically, when the slider 35 is moved in the direction opposite to the finger pull ring 37, the buckling prevention pipe 117 prevents the occurrence of buckling of the manipulation wire 21, thereby enabling the stable manipulation of the manipulation wire 21.

In the base end side of the guide pipe 105, there are provided an O-ring 103 and a washer 101. The washer 101 is locked by a stepped part 33a of the manipulation unit body 33 to restrain the axial movement of the O-ring 103. The inner diameter of the washer 101 is smaller than the outer diameter of the O-ring 103 to prevent the O-ring 103 from escaping to the rear side. In addition, a gap is provided between the washer 101 and the guide pipe 105, and a gap is also provided between the O-ring 103 and the guide pipe 105.

The buckling prevention pipe 117 is inserted through the guide pipe 105, the O-ring 103 and the washer 101 with a proper gap for enabling the rotation of the buckling prevention pipe 117. The inner diameter of the guide pipe 105 is determined as a size that enables the insertion of the buckling prevention pipe 117, and provides a little clearance for the manipulation wire 21. The guide pipe 105 and the fixing clasp 107 are configured to be rotatable in relation to each other. The inner diameter of the O-ring 103 and the outer diameter of the buckling prevention pipe 117 have a relationship in size such that they are rotatable around the axis, and frictional resistance is produced in the axial direction. The parts depicted by dotted lines in FIG. 13 indicate welded parts.

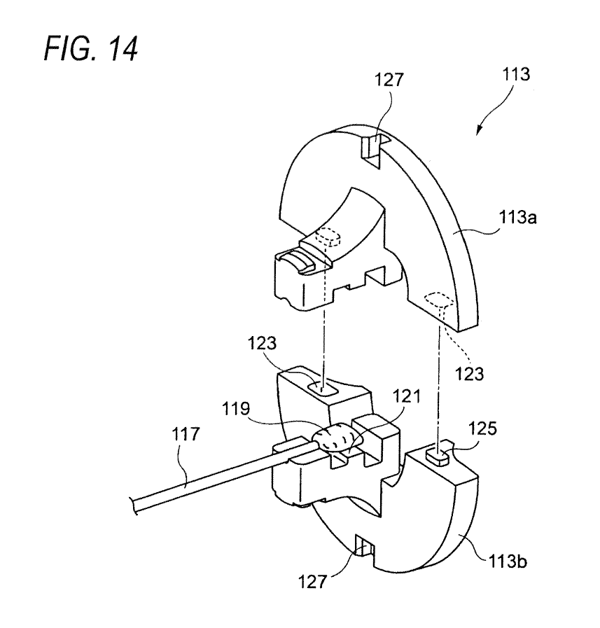

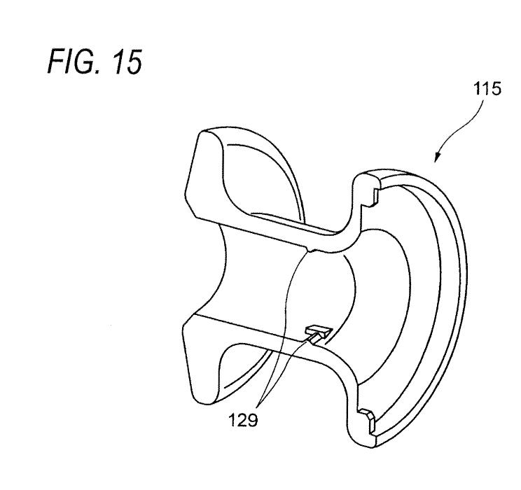

FIG. 14 is an exploded perspective view of a manipulation wire anchor for anchoring the manipulation wire 21 to the slider 35, and FIG. 15 is a sectioned perspective view of the slider body.

The slider 35 is comprised of a slider body 115 and a manipulation wire anchor 113 configured in a halved structure. The slider 35 is fabricated by combining the manipulation wire anchor 113 with the slider body 115 fitted in the manipulation unit body 33 across the slitting section 99 (see FIG. 12) and press-fitting and fixing the manipulation wire anchor 113 to the slider body 115. The slider body 115 and the manipulation wire anchor 113 combined thereby become slidable along the slitting section 99.

As illustrated in FIG. 14, the manipulation wire anchor 113 consists of a top wire anchor 113a and a bottom wire anchor 113b, each of which is substantially semi-circular. The top wire anchor 113a and the bottom wire anchor 113b have the same shape obtained by halving the manipulation wire anchor 113. Both the anchors 113a and 113b have a recess 121 for accommodating a caulking clasp 119 (illustrated in the drawing as a flat shape after mashed) fixedly attached to the rear end of the buckling prevention pipe 117. The top wire anchor 113a and the bottom wire anchor 113b are combined with each other with depressions 123 and protrusions 125. In addition, the bottom wire anchor 113b has a pair of recesses 127, 127 which are engaged with a pair of latching claws 129, 129 of the slider body 115 illustrated in FIG. 15 to prevent relative rotation thereof as well as to conduct positional alignment thereof.

The buckling prevention pipe, the wire and the caulking clasp are integrated with each other by fitting the buckling prevention pipe 117 over the wire, fitting the caulking clasp 119 over the buckling prevention pipe 117, and then caulking the caulking clasp with a pressing tool or the like. The caulking clasp 119 is turned to a non-circular shape through the caulking process, and its relative rotation in relation to the manipulation wire anchor 113 is blocked. As a result, the buckling prevention pipe 117 is fixed to the manipulation wire anchor 113 together with the manipulation wire 21 inserted through the buckling prevention pipe 117.

When performing manipulation by the handle manipulation unit 17 configured as described above, a thumb is inserted into the finger pull ring 37, and the index finger and the middle finger of the same hand are positioned to grasp the slider 35 therebetween. In addition, in order to rotate the clip unit around the axis, the manipulation unit body 33 is rotated.

The rotation of the manipulation unit body 33 is transmitted to the slider 35 and the manipulation wire 21 from the manipulation unit body 33, as illustrated in FIG. 13. In addition, since the manipulation unit body 33 and the fixing clasp 107 are rotatably fixed, the rotation of the manipulation unit body 33 is not transmitted to the sheaths 19, 15 including the fixing clasp 107. That is, by rotating the manipulation unit body 33, the manipulation wire 21 is rotated but the base end sheath 19 and the front end sheath 15 are not rotated.

<Front End Sheath>

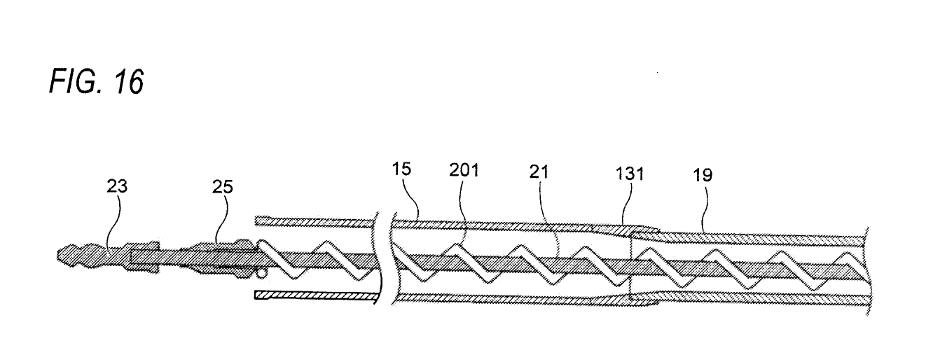

FIG. 16 is a cross-sectional view of the front end sheath. At the base end of the front end sheath 15, the front end part of the base end sheath 19 is welded via a connection ring 131. The inner diameter of the front end sheath 15 is slightly larger than the outer diameter of the outer fastening ring 29 (see FIG. 9), so that the outer fastening ring 29 can be accommodated in the front end sheath 15. The manipulation wire 21 extending from the base end sheath 19 is inserted through the front end sheath 15, and protrudes to the outside toward the front side from the front end sheath 15. At the front end part which is the distal end of the manipulation wire 21, a hook 23 and a guide bead 25 are fixed through soldering or laser welding. As a result, when the slider 35 (see FIG. 12) is slid in the axial direction in relation to the manipulation unit body 33, reciprocating actions are performed following the slide action so as to push the manipulation wire 21 out of the front end sheath 15 to the front side and to return the manipulation wire 21 to the front end sheath 15.

In addition, in the inside of the front end sheath 15 or the base end sheath 19, a retainer coil (a retainer member) 201 is arranged between the front end sheath 15 or the base end sheath 19 and the manipulation wire 21. The retainer coil is formed of a highly wear-resistant hard metal, such as a stainless steel, and is formed in a circular cross-section. By interposing the retainer coil 201 between the inner periphery of the front end sheath 15 or the base end sheath 19 and the manipulation wire 21, the front end sheath 15 or the base end sheath 19 and the manipulation wire 21 do not directly contact with each other and contact with the retainer coil 201 with a small contact area.

For this reason, resistance against the reciprocation of the manipulation wire 21 in the front end sheath 15 or the base end sheath 19 and resistance against the rotation of the manipulation wire 21 may be reduced, and the manipulation wire 21 may be manipulated more smoothly with excellent responsiveness. By determining the hardness of the retainer coil 201 to be substantially equal to that of the manipulation wire 21, the abrasion caused by the sliding movement between the retainer coil 201 and the manipulation wire 21 may be suppressed.

The front end part of the retainer coil 201 is fixedly attached to the guide bead 25, and the retainer coil 201 extends to the handle manipulation unit side. As the retainer coil 201 is arranged in the front end side of the front end sheath where the clip unit is mounted, it is possible to reduce contact resistance in relation to the front end part of the front end sheath which has a lot of opportunities to be curved. For this reason, even if the front end sheath 15 is complicatedly curved, the reciprocation in the axial direction and rotation of the manipulation wire 21 can be smoothly performed. In addition, by fixedly attaching the retainer coil 201 to the guide bead 25, the arranging place of the retainer coil 201 will not be deviated from the front end of the manipulation wire 21.

In addition, the rear end part of the retainer coil 201 is formed as a free end that is not fixed to the front end sheath 15 and the manipulation wire 21. Due to this, the retainer coil 201 becomes extensible within the front end sheath 15, and the rotation of the free end is not restrained. Accordingly, a following performance, such as a curving behavior, can be enhanced.

It is more desirable for the retainer coil 201 to be in a loosely wound condition in which adjacent coil parts are spaced from each other rather than in a closely wound condition in which adjacent coils contact with each other. By forming the retainer coil 201 in the loosely wounded condition, it is possible to further reduce the contact area, thereby further reducing contact resistance. In addition, when cleaning the inside of a sheath, the circulation of cleaning liquid may be facilitated, and the cleaning performance may be improved.

The winding direction of the retainer coil 201 is preferably in a reverse direction to the winding direction for the densely wound coils of the front end sheath 15 and the base end sheath 19. In addition, the winding direction of the retainer coil 201 is preferably in a reverse direction to the winding direction of the stranded wire of the manipulation wire 21. In such a case, it is possible to prevent the retainer coil 201 from penetrating into gaps between strands in the inner surface of a sheath member or gaps between strands of the manipulation wire 21 to increase manipulation resistance. As a result, the twisting characteristics of the retainer coil 201 and the manipulation wire 21 may be averaged to remove unevenness in curving characteristic.

<Hook>

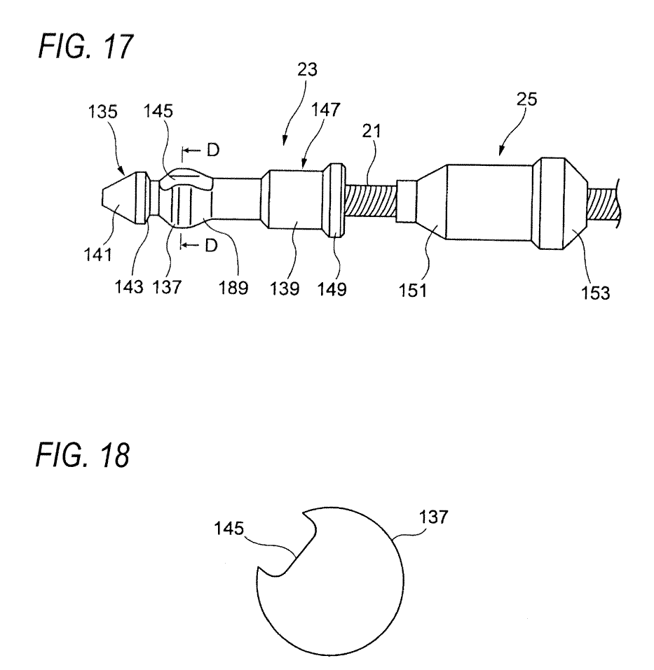

FIG. 17 is a front view of the hook 23 and the guide bead 25 fixed to the front end of the manipulation wire 21, and FIG. 18 is a cross-sectional view taken along line D-D of FIG. 17.

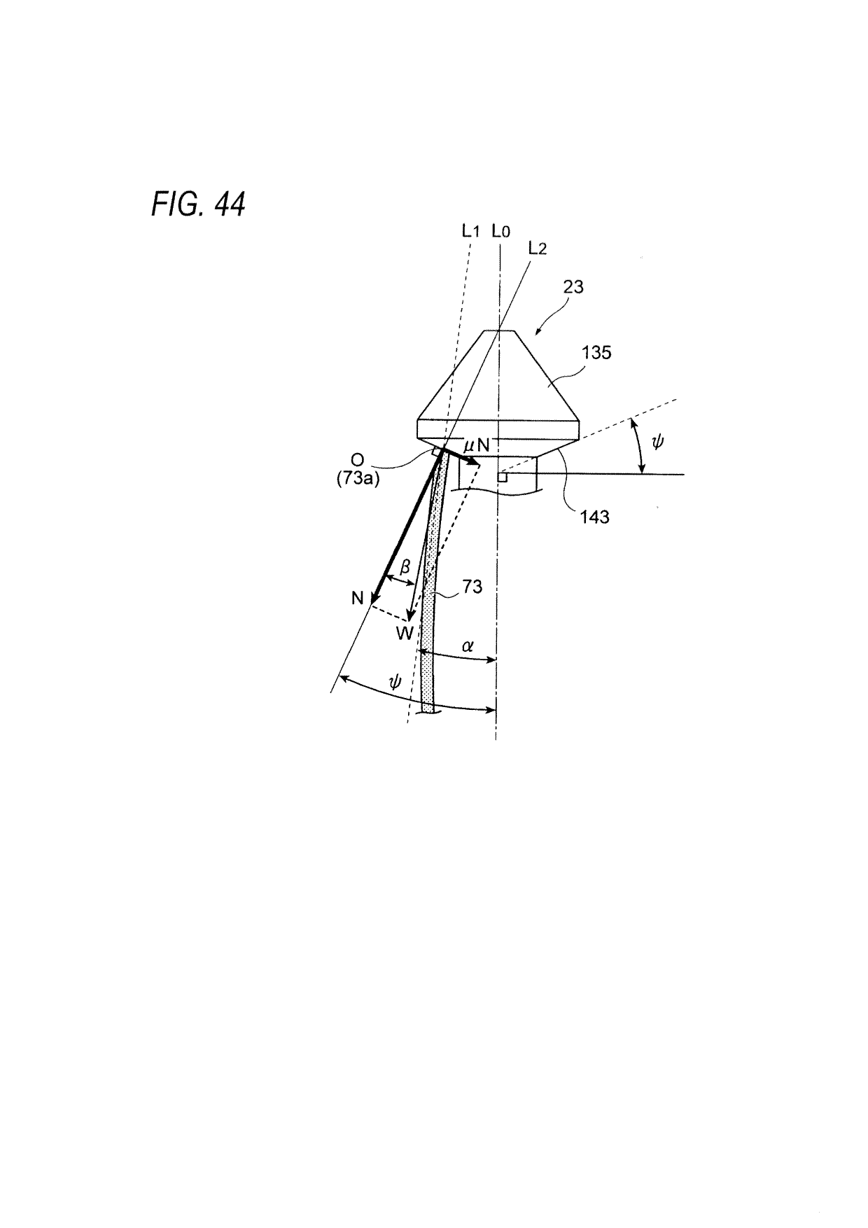

The hook 23 fixed to the front end of the manipulation wire 21 through soldering or laser welding is configured to have the maximum outer diameter smaller than the inner diameter of the outer fastening ring 29 (see FIG. 9) to be capable of being inserted into the outer fastening ring 29. The hook 23 has, from the front end thereof, a hook part 135, an under-head expanded-diameter part (expanded-diameter part) 137, and a rear end expanded-diameter part 139, which are integrally formed. The hook part 135 has a front end taper part (front side inclined surface) 141 and an under-head taper part (rear side inclined surface) 143 formed in this order from the front end to the rear side of the hook 23. The sectional area of the front end taper part 141 perpendicular to the axis Lo of the hook 23 (See, FIG. 44) is gradually increased from the front end to the rear side of the hook 23. And, the sectional area of the rear side inclined surface perpendicular to the axis Lo of the hook 23 (See, FIG. 44) is gradually reduced from the front end to the rear side of the hook 23. The inclined angle of the under-head taper part 143 is designed as an angle of, for example, 90 degrees to 135 degrees in relation to the axial direction, and deforms the J-shaped claw parts 73 of the connection tail section 43, which is engaged with the under-head taper part 143, to control the magnitude of force when releasing the engagement. The cross-section of the hook 23 is not limited to the circular shape and may have a different shape, such as a pyramid shape or the like.

The under-head expanded-diameter part 137 has front and rear sides, each of which is formed in a taper shape, and a key recess 145 engaged with the J-shaped claw parts 73 provided in the connection tail section 43 of the clip body 27 (see FIG. 4) is formed at a portion of the outer periphery of the under-head expanded-diameter part 137. When the hook 23 and the connection tail section 43 of the clip body 27 are connected, the under-head expanded-diameter part 137 maintains the coaxiality between the hook 23 and the connection tail section 43, and secures the stability of engagement. In addition, the rear end expanded-diameter part 139 includes a small diameter part 147, and a large diameter part 149 formed at the rear end of the small diameter part 147.

<Guide Bead>

The guide bead 25 is spaced from the hook 23 in the rear side of the hook 23 and fixed to the manipulation wire 21 by soldering or laser welding. Since the hook 23 and the guide bead 25 are spaced from each other and fixed to the manipulation wire 21, the manipulation wire 21 may be curved therebetween and the degree of freedom in mutual movement may be improved. At the front end of the guide bead 25, a front end taper part 151 is formed, and at the rear end, a rear end taper part 153 is formed. The front end taper part 151 has an effect of guiding the guide bead 25 into the outer fastening ring 29 to be smoothly inserted into the outer fastening ring 29. The rear end taper part 153 has an effect of guiding the guide bead 25 to the front end sheath 15 to be smoothly accommodated in the front end sheath 15 when the clip unit 13 is pulled into the front end sheath 15.

The rear end taper part 153 is set to have a length that is exposed to the rear side from the rear end of the outer fastening ring 29 when the hook 23 and the clip unit 13 are connected. Due to this, the rear end taper part 153 can be smoothly introduced into the front end sheath 15 without causing the front end part of the front end sheath and the rear end part of the clip unit 13 to be interfered with each other even if the entirety of the clip unit 13 has completely protruded to the outside from the front end sheath.

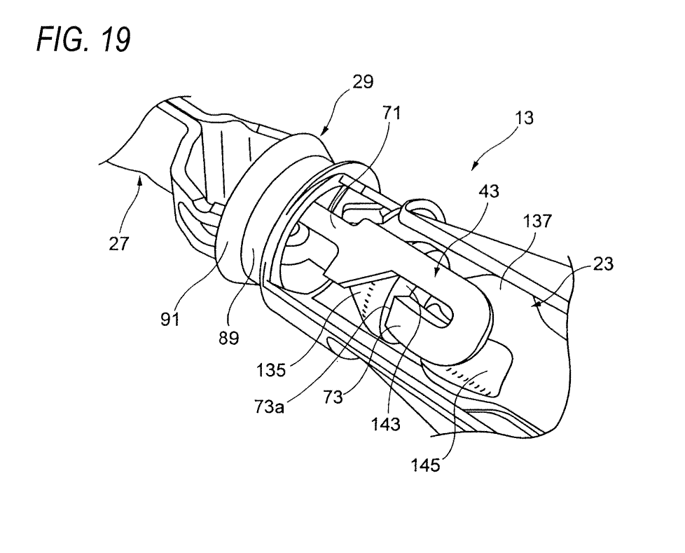

FIG. 19 is a perspective view illustrating, partially in cut-away, the clip body and the hook in the state where they are engaged with each other.

When the hook 23 and the clip unit 13 are connected, the clip body 27 is restrained in rotating position around the axial direction since the J-shaped claw parts 73 of the connection tail section 43 are engaged in the key recess 145. In addition, the front ends 73a of the J-shaped claw parts 73 are engaged with the under-head taper part 143, thereby determining the axial position of the clip body 27. In this state, since the key recess 145 is engaged with the connection tail section 43 (J-shaped claw parts 73) of the clip body 27, the torque of the manipulation wire 21 can be transmitted to the clip body 27.

<Clip Case>

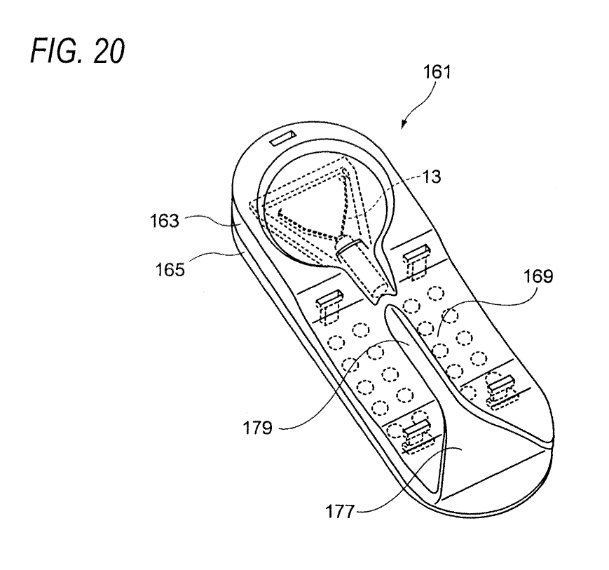

In the clip manipulation device 11 configured as described above, the clip unit 13 is mounted on the hook 23 provided at the front end of the manipulation wire 21, and a biological tissue is ligated by a pair of the arm parts of the clip unit 13. The clip unit 13 is accommodated in a clip case 161 in advance, which is illustrated in FIG. 20 as a perspective view, and the clip unit 13 is mounted on the hook 23 by inserting the hook 23 at the front end of the manipulation wire 21 into the case from the accommodated state.

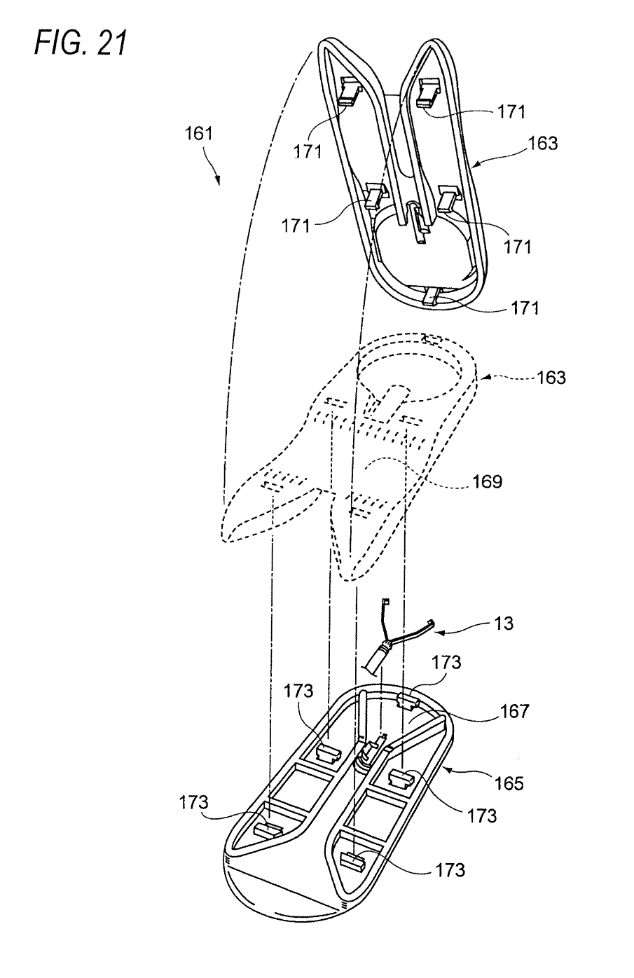

An exploded perspective view of the clip case is illustrated in FIG. 21. The clip case 161 is comprised of a top case 163 and a bottom case 165, and is configured by combining the top case 163 and the bottom case 165. The clip unit 13 is located in a clip receiving chamber 167 formed between the top case 163 and the bottom case 165. The top case 163 and the bottom case 165 are integrated by engaging a plurality of engagement claws 171 formed in the top case 163 with claw engagement parts 173 formed in the bottom case 165.

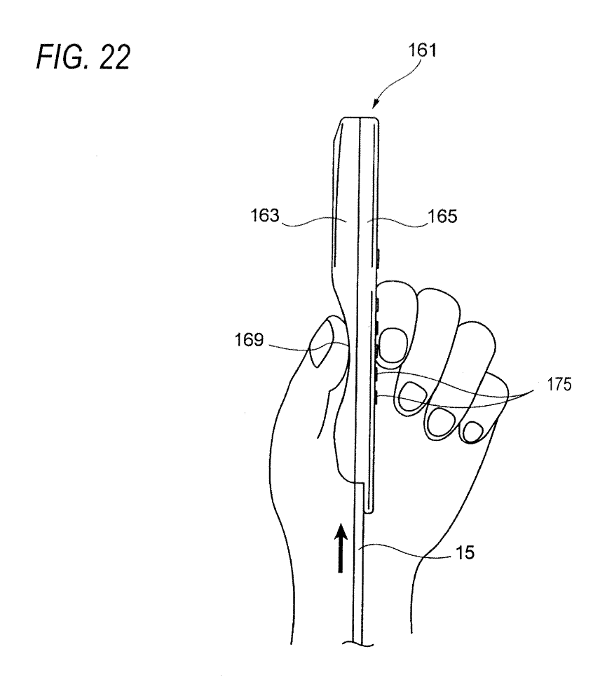

A side view of the clip case is illustrated in FIG. 22. The top case 163 has a sheath press part 169 narrowly formed in the thickness direction. In addition, an anti-slipping uneven pattern 175 is formed on the bottom case 165 at a position corresponding to the sheath press part 169, thereby enhancing a grip performance when the clip case 161 is grasped.

<Mounting of Clip Unit>

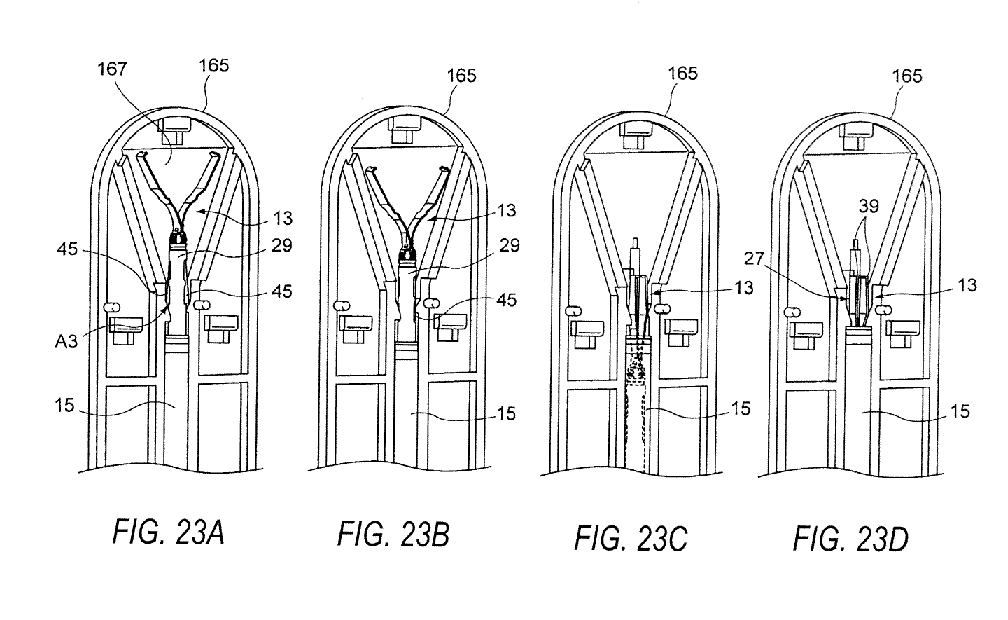

A sequence of mounting the clip unit of the clip case to the hook is illustrated in FIGS. 23A to 23D. As illustrated in FIG. 23A, the clip unit 13 is arranged in the clip receiving chamber 167 in advance.

In order to mount the clip unit 13 to the clip mounting device, the front end sheath 15 is firstly inserted into a sheath insertion part 179 from a sheath guide part 177 of the clip case 161, which are illustrated in FIG. 20. In addition, the front end sheath 15 is pushed into the sheath insertion part 179 until the front end of the front end sheath 15 is abutted against the end of the inside of the sheath insertion part 179. In this state, the clip case 161 is gripped in the thickness direction while pressing the front end sheath 15 against the clip case 161 as illustrated in FIG. 22.

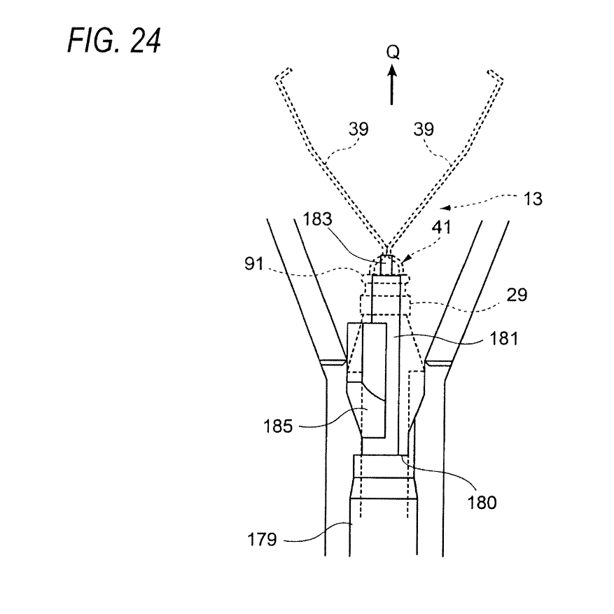

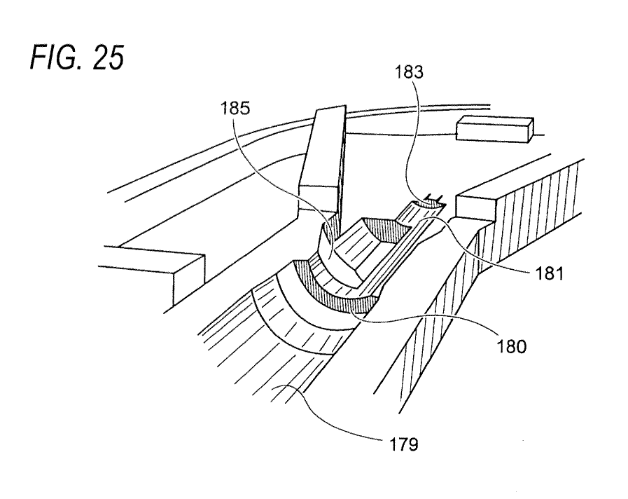

The part indicated by arrow A3 of the bottom case 165 in FIG. 23A is illustrated in FIG. 24 as an enlarged view and is illustrated in FIG. 25 as a perspective view, respectively.

As illustrated in FIGS. 24 and 25, the front end of the front end sheath 15 is pushed into the sheath insertion part 179 to a position where the front end abuts against the sheath abutting part 180. Then, the outer fastening ring 29 is positioned such that the taper section 91 of the front end abuts against a fastening ring abutting part 183 in a state where the outer fastening ring 29 is laid in a fastening ring retaining part 181 of a concavely curved shape. As a result, the clip unit 13 is prevented from getting out in the direction indicated by arrow Q in FIG. 24. That is, even if a forward pushing force is applied to the clip unit 13 by inserting the hook, the relative position of the clip unit 13 and the clip case 161 is not changed. For this reason, the clip unit 13 is prevented from coming into contact with and being damaged by the clip case 161.

Since the outer periphery of the outer fastening ring 29 is retained in the fastening ring retaining part 181 corresponding to the outer periphery, the outer fastening ring 29 is precisely retained within the clip case 161, and the outer fastening ring 29 may be arranged without axial deviation. Therefore, the front end part of the front end sheath 15 and the rear end part of the outer fastening ring 29 will not interfere with each other when the front end sheath 15 is inserted into the clip case 161.

In addition, the flap parts 45 of the inner fastening ring 31 come into contact with the flap guide taper part 185 such that the clip unit 13 laid in the clip case 161 is biased to the base end side not to be disengaged. As a result, the clip unit 13 will not escape from the clip case 161 by the self-weight of the clip unit 13, vibration at the time of transportation, and handling of the clip case.

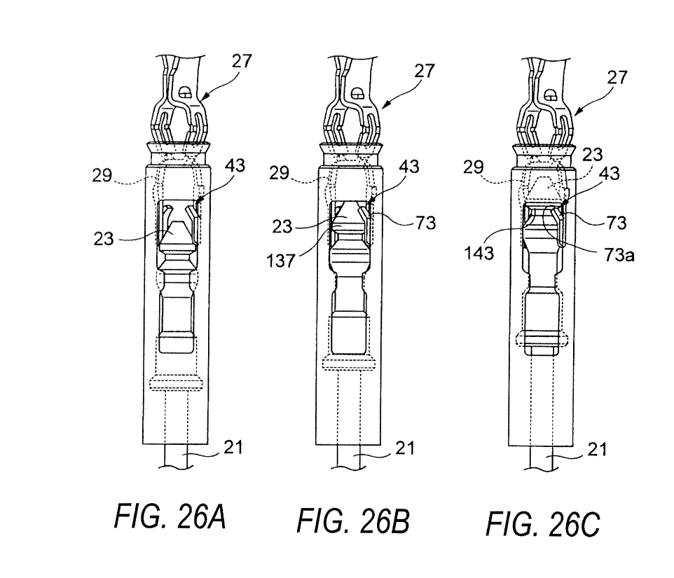

Then, the slider 35 of the handle manipulation unit 17 is moved forward, and the manipulation wire 21 is extended, so that the hook protrudes to the outside from the front end of the front end sheath 15. FIGS. 26A to 26C are explanatory views illustrating the hook from the state where it is inserted into the connection tail section 43 of the clip body 27 to the state where it is connected to the connection tail section 43 in a step-by-step manner. The hook 23 starts to be engaged with the connection tail section 43 as illustrated in FIG. 26A, then the J-shaped claw parts 73 are moved to pass the under-head expanded-diameter part 137 of the hook 23 as illustrated in FIG. 26B, and then the front ends 73a of the J-shaped claw parts 73 are engaged with the under-head taper part 143 of the hook 23 as illustrated in FIG. 26C, thereby completing the connection between the hook 23 and the clip body 27.

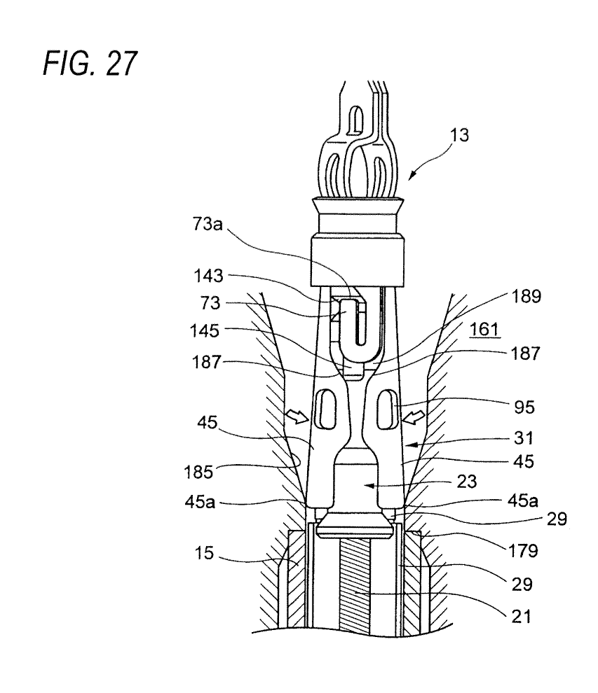

Next, the slider 35 of the handle manipulation unit 17 is moved backward to pull the manipulation wire 21 backward. As such, as illustrated in FIG. 23B, the flap parts 45 extending radially outward from the outer fastening ring 29 are closed. FIG. 27 is an enlarged explanatory view illustrating the flap parts while they are being closed. When the manipulation wire 21 is pulled backward, the front ends 73a of the J-shaped claw parts 73 are abutted against the under-head taper part 143 of the hook part 135, whereby the pulling force is transmitted to the clip unit 13. When the clip unit 13 is pulled out to the base end side in relation to the clip case 161, the free ends 45a of the flap parts 45 abut against the flap guide taper part 185 of the clip case 161, thereby gradually reducing the opening extent, and finally closing the opening to the extent of the outer diameter of the outer fastening ring 29.

When the free ends 45a of the flap parts 45 are closed to the outer diameter of the outer fastening ring 29, the clip unit 13 is inserted into the front end sheath 15 as illustrated in FIG. 23C. The force at the time of closing the flap parts 45 is set to be sufficiently smaller than that for deforming and introducing the loop parts 47 of the base end section 41 (see FIG. 4) of the clip body 27 into the outer fastening ring 29. For this reason, the flap parts 45 are securely closed.

In addition, when the flap parts 45 are closed, hook abutments 187 of the flap parts 45 abut against a flap abutting taper part 189 of the hook 23. Due to this abutting of the hook abutments 187, the relative movement between the hook 23 and the inner fastening ring 31 is restrained, and the reciprocating movement by the manipulation wire 21 in the axial direction may be directly transmitted to the inner fastening ring 31. That is, the position for transmitting force from the hook 23 to the clip unit 13 after the flap parts 45 are closed is changed from the engagement position of the J-shaped claw parts 73 and the under-head taper part 143 of the hook 23 to the engagement position of the hook abutments 187 and the flap abutting taper part 189 of the hook 23.

When the manipulation wire 21 is pulled backward, the clip unit 13 is stored in the front end sheath 15 as illustrated in FIG. 23D. When the clip unit 13 is stored, the arm parts 39 of the clip body 27 are guided to the ring part 77 of the inner fastening ring 31 (see FIG. 6) and inserted into the front end sheath 15 while being closed.

As such, the clip unit 13 is mounted to the clip manipulation device 11 illustrated in FIG. 1.

<Manipulation for Ligation>

Next, manipulation for ligating a biological tissue by the ligation device 100 illustrated in FIG. 1 will be described.

(Insertion of Sheath into Forceps Channel)

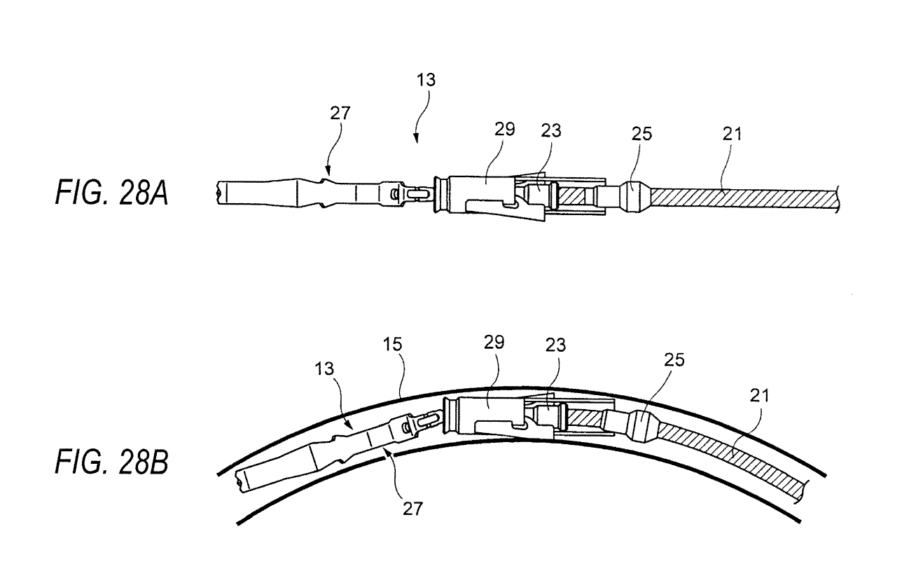

An operator of an endoscope inserts the front end sheath 15 into a forceps channel which is a treatment instrument insertion passage of the endoscope in a state where the clip unit 13 is stored in the front end sheath 15 of the ligation device 100. In such a case, the connected form of the clip body 27 and the outer fastening ring 29 is changed to a curved state illustrated in FIG. 28B by following the curve of the front end sheath 15 from the linear state illustrated in FIG. 28A. Since the hook 23 and the guide bead 25 are spaced from each other and fixed to the manipulation wire 21, the manipulation wire 21 can be curved between the hook 23 and the guide bead 25, and the degree of freedom in mutual movement can be enhanced.

The outer fastening ring 29 is formed from a hard metal material. For this reason, the longitudinal length of the outer fastening ring 29 determines the facilitation of insertion in relation to the endoscope, and it is desirable that the entire length of the outer fastening ring 29 is short.

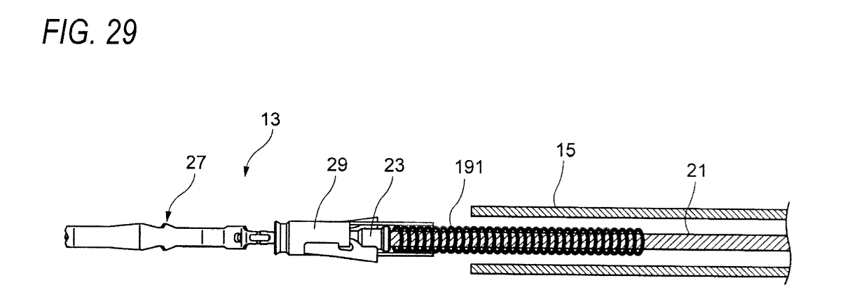

As illustrated in FIG. 29, the guide bead 25 may be replaced by a guide spring 191 arranged to the outside of the manipulation wire 21 in the rear side of the outer fastening ring 29. By setting the guide spring 191 is a length that makes the rear end of the guide spring 191 be always inserted into the front end sheath 15 in the entire stroke area of the reciprocating movement of the manipulation wire 21 in the axial direction, it is possible to secure the following performance for curving and a guidance performance to the front end sheath 15.

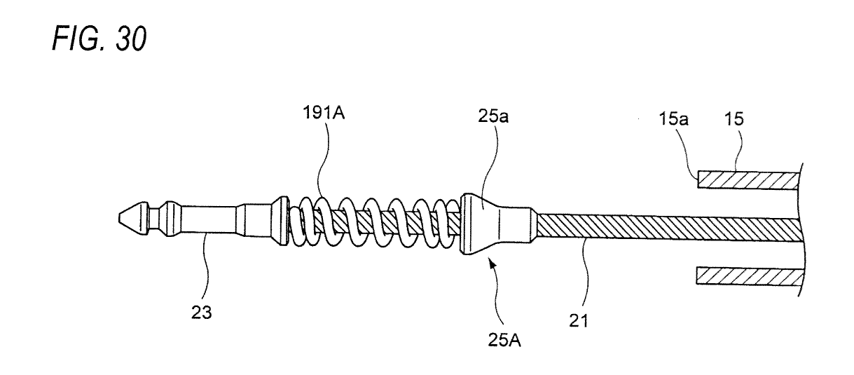

As illustrated in FIG. 30, a guide bead 25A having a rear end taper part 25a may be provided at the rear end of the guide spring 191A. The guide bead 25A is fixed to the manipulation wire 21 by soldering or welding. The guide spring 191A is arranged to be free between the hook 23 and the guide bead 25A without being biased. With this arrangement, even if the guide spring 191A is set in a length that makes the rear end thereof project from the front end sheath 15, the rear end of the guide spring 191A can be introduced into the front end sheath 15 without interference between the front end 15a of the front end sheath 15 and the guide spring 191A. Accordingly, the following performance for curving and the guidance performance to the front end sheath 15 can be secured and the entire length of the guide spring 191A can be set shortly. Therefore, the flexibility can be further enhanced.

(Clip Protrusion from Front End Sheath)

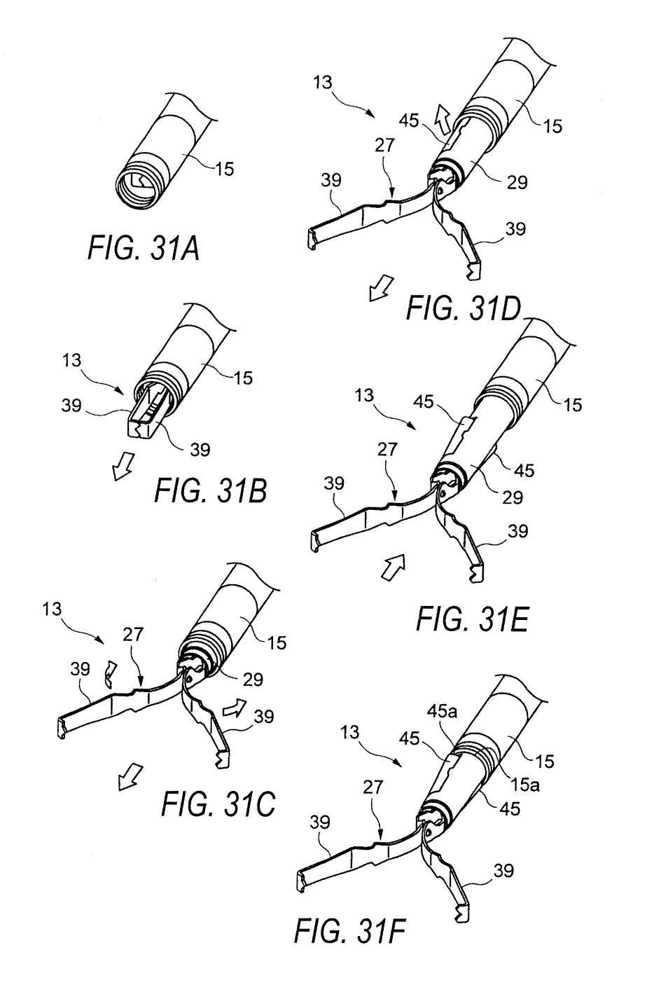

FIGS. 31A to 31F illustrate the arm parts of the clip unit until they are expanded from the front end sheath.

As illustrated in FIG. 31A, the clip unit 13 protrudes to the outside from the front end of the front end sheath 15 by continuously feeding the manipulation wire toward the front side by manipulation from the handle manipulation unit in the state where the clip unit is accommodated in the front end sheath 15. FIG. 31B illustrates the clip unit 13 when the arm parts 39 start to protrude from the front end sheath 15.

When the clip unit 13 protrudes to the outside from the front end sheath 15, the arm parts 39 are gradually expanded as illustrated in FIG. 31C. Then, when the flap protruding holes 93 of the outer fastening ring 29 completely get out of the front end sheath 15 as illustrated in FIG. 31D, the flap parts 45 are elastically returned from the flap protruding holes 93 and hence individually opened diametrically outward. The flap parts 45 are maintained at a predetermined opening angle where each of the flap opening prevention taps 95 is engaged with one side 85a of each one of the flap locking holes 85 (see FIG. 11).

Then, as illustrated in FIG. 31E, the clip unit 13 further protrudes from the front end sheath until the flap parts 45 are certainly opened, and as illustrated in FIG. 31F, the clip unit 13 is returned to the rear side by pulling the manipulation wire to the base end side. At this time, the free ends 45a of the flap parts 45 are stopped at a position where they abut against the front end 15a of the front end sheath 15.

That is, the flap parts 45 function as a stopper for determining the axial positions of the clip unit 13 and the front end sheath 15. The diametrically outward opening angle of the flap parts 45 is determined by the designed sizes of the outer fastening ring 29 and the inner fastening ring 31 rather than the front end sheath 15, and hence the opening angle is fixed. For this reason, even if an axial deviation or inclination of the clip unit 13 and the front end sheath occurs, the free ends 45a of the flap parts 45 certainly abut against the front end 15a of the front end sheath 15, thereby functioning as a stopper at a correct position.

(Opening/closing Action of Arm Parts)

Next, the opening/closing action of the arm parts for gripping a biological tissue will be described.

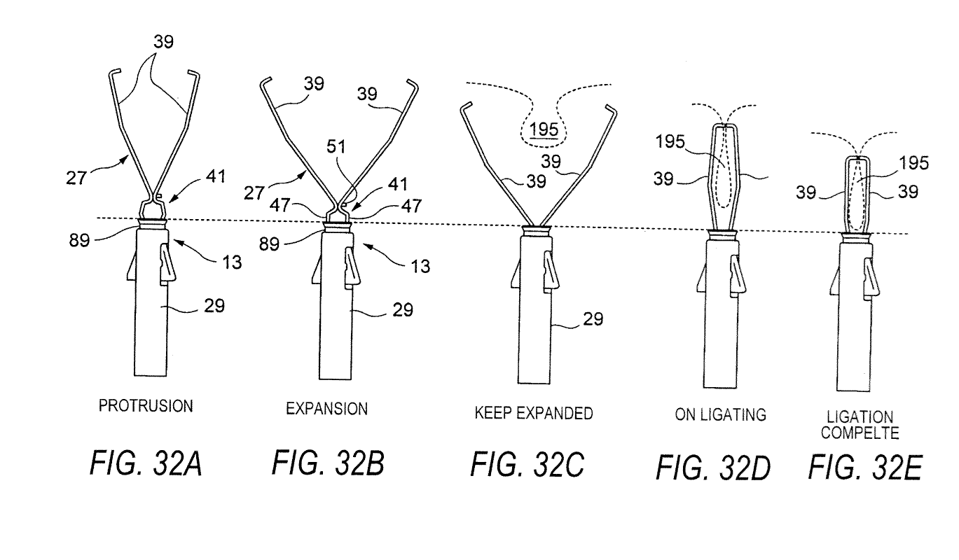

FIGS. 32A to 32E illustrate the actions of the arm parts of the clip unit, from expanding after having protruded to the outside from the front end sheath to ligating a biological tissue, in a step-by-step manner.

Since the arm parts 39 of the clip body 27 are closed when the clip unit 13 stored in the front end sheath 15, the clip unit 13 tends to be closed, thereby reducing the opening angle. The plastic deformation of the base end section 41 may compensate for the reduction of the opening angle.

When the base end section 41 of the clip body 27 illustrated in FIG. 32A is inserted into the constricted spot 89 of the outer fastening ring 29 by being pulled by the manipulation wire, the loop parts 47, 47 are gradually narrowed as illustrated in FIG. 32B, and hence the base end section 41 is plastically deformed. That is, the outer diameter of the base end section 41 of the clip body 27 is larger than the inner diameter DS (see FIG. 9) in the major axis side in the constricted spot 89 of the outer fastening ring 29. For this reason, when the base end section 41 of the clip body 27 is pulled into the outer fastening ring 29, the loop parts 47, 47 of the base end section 41 are pressed and deformed from the opposite sides thereof.

As the loop parts 47, 47 of the base end section 41 are deformed, the arm parts 39, 39 of the clip body 27 perform expansion movement using a deviation prevention claw 51 as a fulcrum point. Accordingly, as illustrated in FIG. 32B, the arm parts 39, 39 are widely expanded as illustrated to have an opening size that is capable of gripping a sufficient amount of tissue.

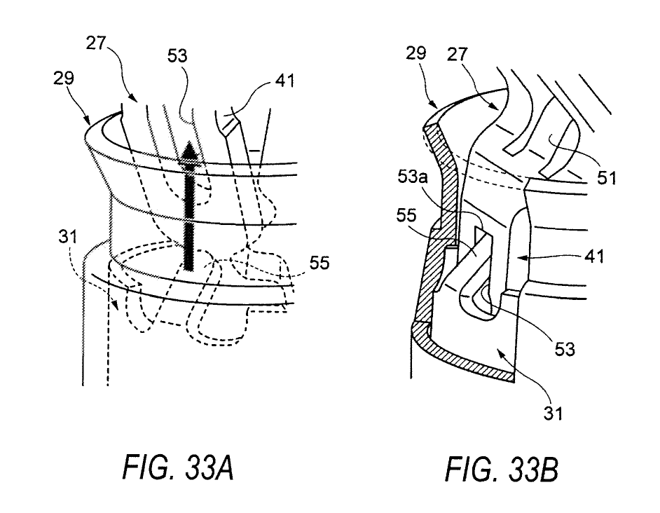

The timing of expanding the arm parts 39, 39 is transmitted to an operator as timing when a resistance against the pulling of the manipulation wire has occurred. That is, an expansion retaining protrusion 55 of the inner fastening ring 31 illustrated in FIG. 33A is introduced into the slit part 53 formed in the base end section 41 of the clip body 27 as the base end section 41 is moved to the base end side (downward in the drawing) by pulling the manipulation wire. When the manipulation wire is pulled again, the expansion retaining protrusion 55 abuts against the front end part 55a of the slit part 53, thereby producing a resistance against the pulling of the manipulation wire as illustrated in FIG. 33B.

With the resistance, the operator may readily recognize that the arm parts 39, 39 are opened at their maximum, and may temporarily stop the pulling operation of the manipulation wire in the state where the arm parts 39, 39 are opened at their maximum.

(Rotation of Clip)

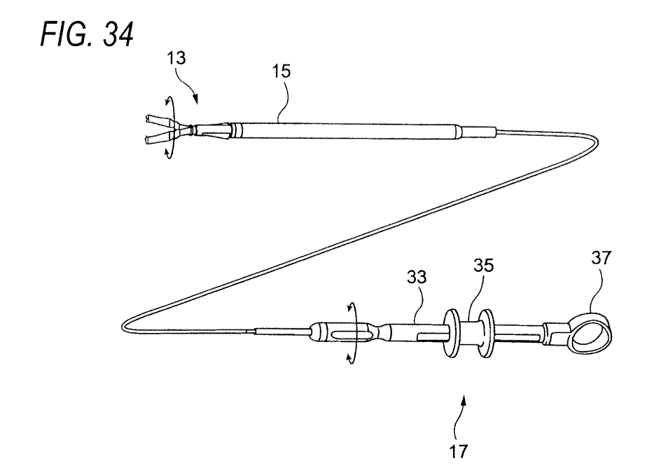

As illustrated in FIG. 32C, the operator moves the clip unit 13 mounted in the front end sheath to a treatment position within a body cavity in the state where the arm parts 39, 39 are kept expanded. Then, the clip unit 13 is rotated according to a treatment direction. As described above, the rotation of the clip unit is performed by rotating the manipulation unit body 33 illustrated in FIG. 34 around the axis

(Starting of Ligation)

Next, as illustrated in FIG. 32C, the operator sets the arm parts 39, 39 to a target treatment position (an affected area 195), and pulls the slider 35 of the handle manipulation unit 17 (see FIG. 34) to the rear side, i.e., to the finger pull ring 37 side. Accordingly, as illustrated in FIG. 32D, the arm parts 39, 39 are closed, and the affected area 195 is gripped. At this time, the bent linear plate parts 57 of the arm parts 39, 39 (see FIG. 4) slidably contact with the inner diameter areas of the major axis (DL) side in the constricted spot 89 of the outer fastening ring 29 (see FIG. 9), and hence the ligation action of the clip is performed.

At this time, in the base end section 41 of the clip body 27 illustrated in FIG. 33, the expansion retaining protrusion 55 formed in the slit part 53 is plastically deformed, so that the engagement between the clip body 27 and the inner fastening ring 31 is released. The expansion retaining protrusion 55 is set to be plastically deformed by a force in the extent of 10 N to 20 N, and the engagement with the inner fastening ring 31 may be readily released with the force applied when gripping the affected area 195 with the arm parts 39, 39.

As a result, the clip body 27 is made to be movable backward again, and by retracting the slider 35 to the finger pull ring 37, the clip body 27 is inserted into the outer fastening ring 29 as illustrated in FIG. 32E.

(Ligation State)

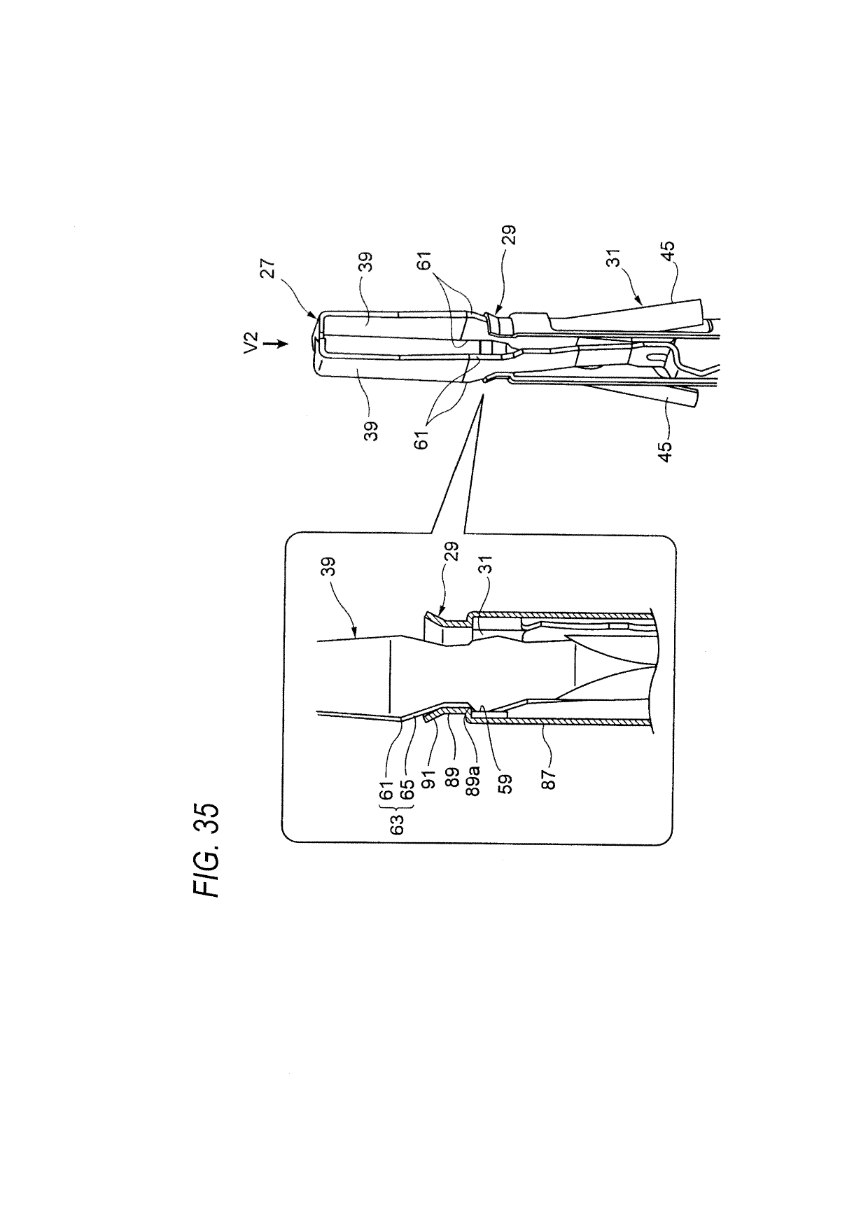

When the arm parts 39, 39 are completely closed, a disengagement prevention protrusion 59 formed widely on a portion of each of the arm parts 39 is locked at the stepped part 89a formed by the constricted spot 89 of the outer fastening ring 29 as illustrated in FIG. 35. As a result, it is possible to prevent the clip body 27 from coming out to the front side of the outer fastening ring 29, and the ligation state by the arm parts 39, 39 will be retained.

In addition, when the clip body 27 is moved backward (downward in the drawing), the taper part 65 of the arm parts 39 and the pull-in prevention protrusions 61 abut against the inner diameter areas of the minor axis DS side of the constricted spot 89 (see FIG. 9), so that the clip body 27 cannot be further pulled into the outer fastening ring 29.

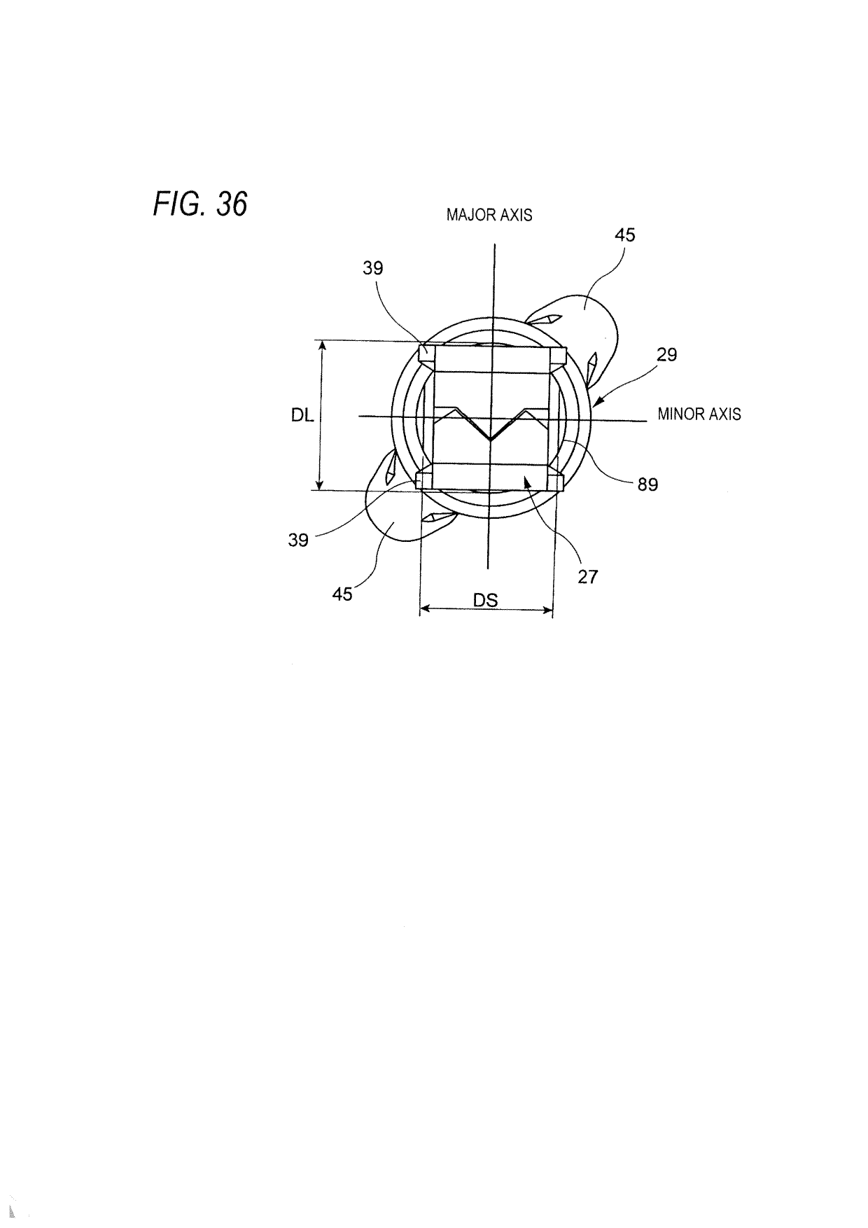

FIG. 36 illustrates a view shown in the direction indicated by arrow V2 in FIG. 35.

As described above, the shape the inside of the constricted spot 89 is formed in an oval shape, in which the diameter of the major axis DL and the diameter of the minor axis DS are different from each other. The major axis of the constricted spot 89 is set to coincide with the expansion direction of the arm parts 39, 39 of the clip body 27. Due to this, the constricted spot 89 abuts against the loop parts 47, 47 of the base end section 41 (see FIG. 4) to control the expansion extent of the pair of arm parts 39. In addition, the minor axis of the constricted spot 89 is set to be parallel to the widthwise direction of the each of the pull-in prevention protrusions 61 on the arm parts 39, 39. Due to this, when the clip body 27 is accommodated in the outer fastening ring 29, the constricted spot 89 engaged with the pull-in prevention protrusions 61 to control the pull-in extent of the clip body 27.

(Release of Clip Unit)

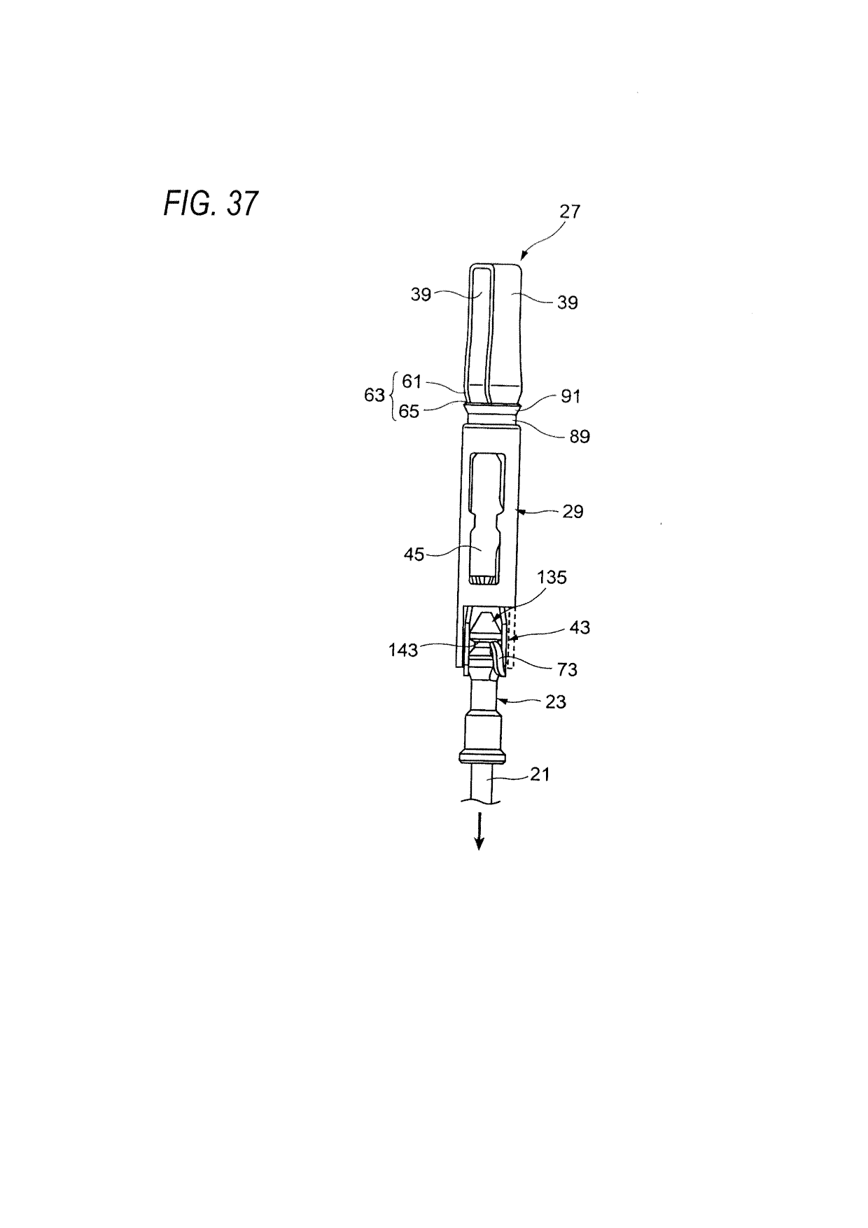

FIG. 37 illustrates, partially in cut-away, the clip unit in the state where the clip unit has completed ligation.

If the pulling force of the hook 23 is further increased in the state where the pull-in prevention protrusions 61 formed on the arm parts 39 of the clip body 27 and the constricted spot 89 of the outer fastening ring 29 are engaged with each other, a tensile force is also applied to the engagement part between the hook 23 and the connection tail section 43 of the clip body 27.

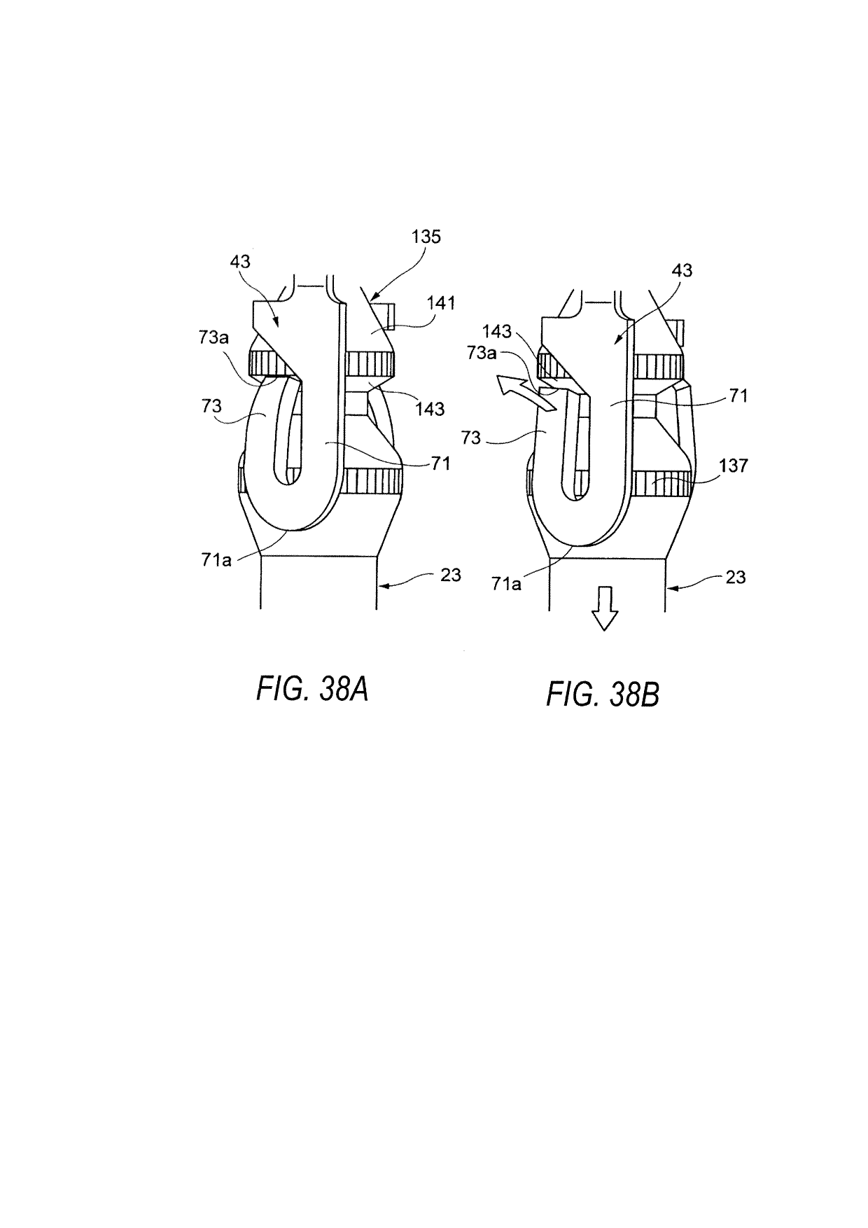

FIG. 38A illustrates the hook and the connection tail section in the engaged state, and FIG. 38B illustrates the hook and the connection tail section in the state of starting to release the engagement. As illustrated in FIG. 38A, the hook part 135 of the hook 23 is positioned such that the front ends 73a of the J-shaped claw parts 73 of the connection tail section 43 abut against the under-head taper part 143 formed on the rear side from the largest outer diameter area of the front end taper part 141. When the hook 23 is returned backward in relation to the connection tail section 43, the J-shaped claw parts 73 are forcibly spread diametrically outward, thereby releasing the engagement with the under-head taper part 143 as illustrated in the FIG. 38B.

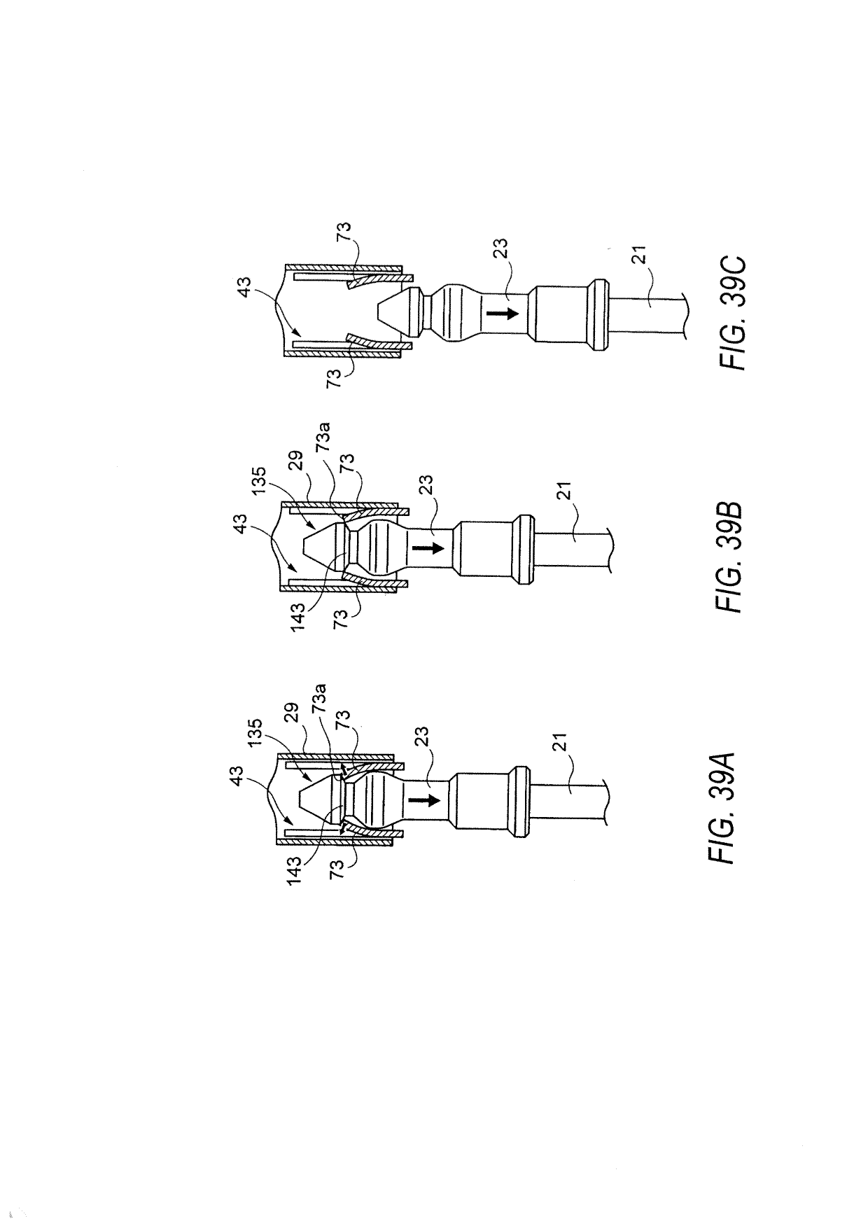

FIGS. 39A to 39C illustrate a process of releasing the J-shaped claw parts from the engagement in a step-by-step manner.

As illustrated in FIG. 39A, when the hook 23 is returned backward from the state in which the front ends 73a of the J-shaped claw parts 73 of the connection tail section 43 abut on the under-head taper part 143, the front ends 73a of the J-shaped claw parts 73 are expanded diametrically outward while being slid on the under-head taper part 143. In addition, when the front ends 73a of the J-shaped claw parts 73 are spread diametrically outward over the maximum outer diameter of the hook part 135 as illustrated in FIG. 39B, the hook 23 is disengaged from the connection tail section 43 and the engagement of the hook 23 and the connection tail section 43 is released as illustrated in FIG. 39C.

Due to this, it is possible to retract the clip manipulation device within a body cavity in the state where the clip unit that has ligated a biological tissue such as an affected area is detained in the body cavity.

<Examples of Other Configurations of Clip Body>

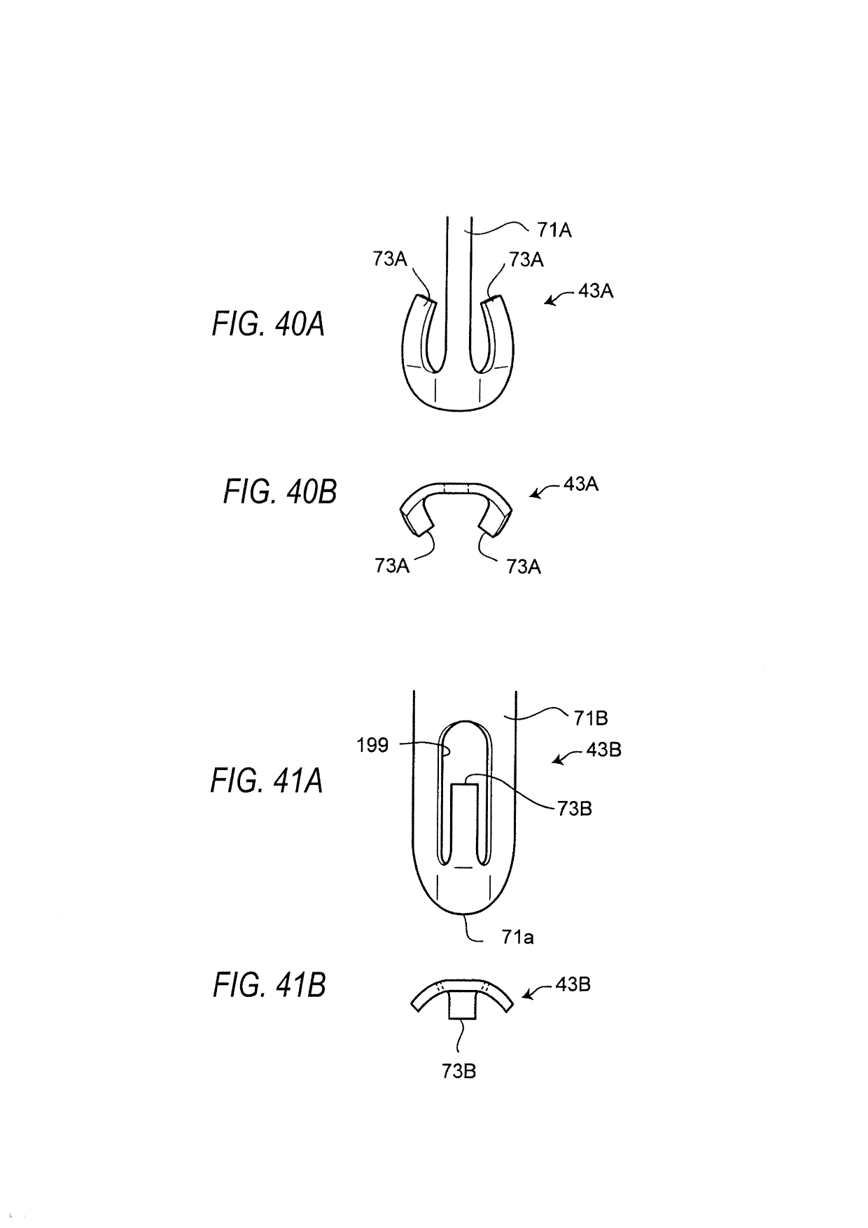

FIGS. 40A and 40B and FIGS. 41A and 41B illustrate different examples of configuration of the connection tail section 43, respectively.

FIG. 40A is a partial configuration view of a connection tail section formed with a pair of engagement claw parts 73A, 73A, and FIG. 40B is a bottom view of FIG. 40A. In this example of configuration, the pair of the engagement claw parts 73A, 73A are provided respectively to extend toward the front side with reference to the rear end 71a of the arm 71A. The engagement claw parts 73A, 73A are inwardly inclined toward the central axis of the hook accommodated by the connection tail section 43A as illustrated in FIG. 40B.

With this arrangement, the engagement claw parts 73A, 73A abut against the rear inclined surface of the hook, whereby the connection strength between the clip unit and the hook can be increased. In addition, the number of the engagement claw parts is not limited two, and three or more engagement claw parts may be provided

In addition, since the loads from the engagement claw parts 73A, 73A are uniformly applied and the bending moment applied to the part 71A is reduced, it is possible to increase the connection strength.

FIG. 41A illustrates a partial configuration view in which an engagement claw part is arranged in an opening formed in an arm 71B, and FIG. 41B is a bottom view of FIG. 41A.

In this example of configuration, an opening 199 is formed in the vicinity of the rear end 71a of the arm 71B, and an engagement claw part 73B is provided in the opening 199 to extend from the rear end 71a side of the opening 199 to the front side. The engagement claw part 73B is inclined inwardly toward the central axis of the hook to the same with the above-described claw parts.

With this arrangement, the engagement claw part 73B may be simply configured. As a result, it is easy to fabricate and assemble.

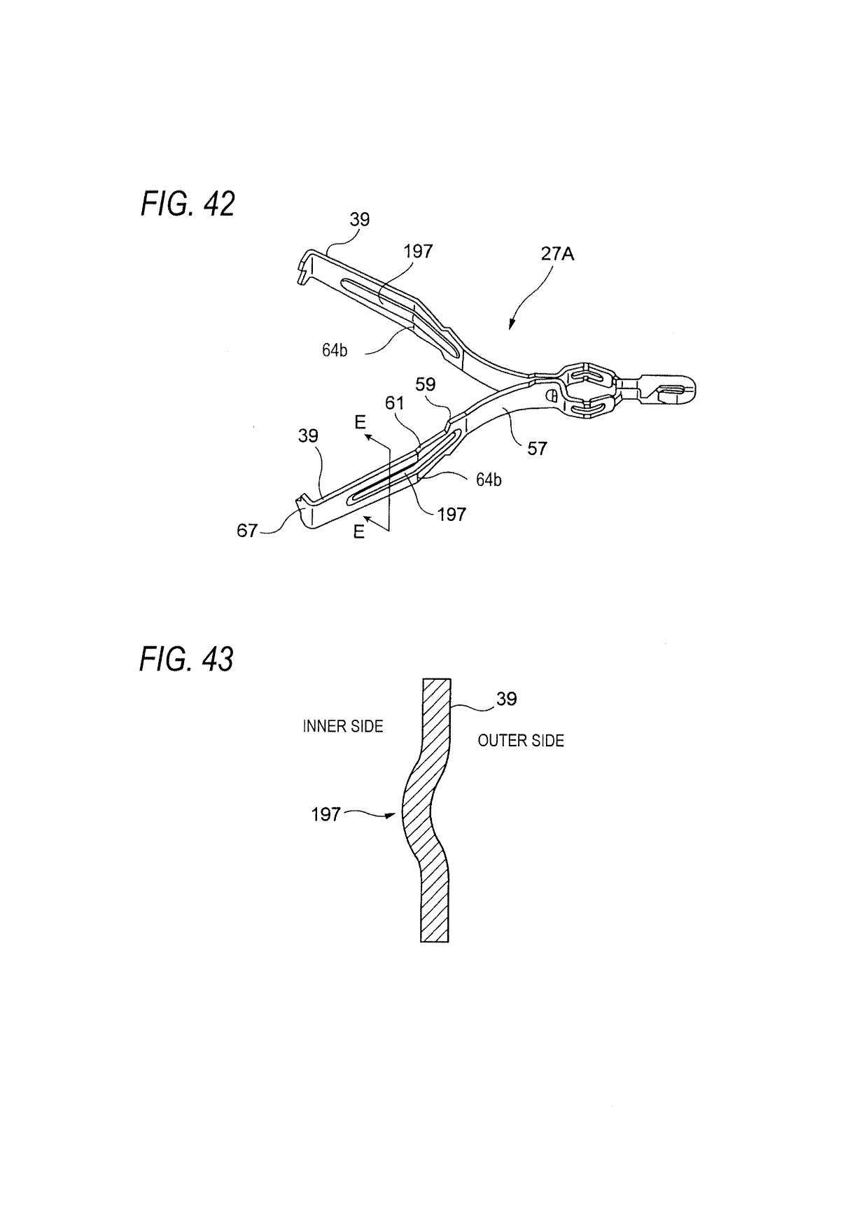

The above-described clip body 27 may be formed by bending a flat metallic plate, and a large force is applied to the arm parts 39, 39 at the time of ligation. Therefore, as illustrated in FIG. 42, a clip body 27A is formed with reinforcement ribs 197, 197 extending in the longitudinal direction with the second bending points 64b as the centers, respectively, to increase the strength of the clip body 27A. The reinforcement ribs 197, 197 are formed to bulge on the inner sides which the pair of the arm parts 39, 39 are opposite to each other.

FIG. 43 is a cross-sectional view taken along line E-E of FIG. 42. Each of the reinforcement ribs 197 is formed by pressing an arm part 39 in the thickness direction of the plate. Since the strength of the clip body 27 is increased by providing the reinforcement ribs 197, 197, a stronger ligation may be performed for a biological tissue, and the arm parts may be formed more narrowly to miniaturize the clip body.

<Description of Resultant Force for Connection Release>

Next, a description will be made for setting a resultant force required for connection release from the state where the front ends 73a of the J-shaped claw parts 73 of the connection tail section 43 abut against the under-head taper part 143 as illustrated in FIG. 39A to the connection release state as illustrated in FIG. 39C.

The state illustrated in FIG. 39A is the state in which the front ends 73a of the J-shaped claw parts 73 start to abut against the under-head taper part 143. The acting forces at that time will be described with reference to FIG. 44. FIG. 44 illustrates acting forces loaded to the abutting point O in the state where the under-head taper part 143 and the front end 73a of a J-shaped claw part 73 start to abut against each other in the hook part 135 of the front end of the hook 23. The J-shaped claw part 73 of the illustrated example abuts against an inclined surface of the under-head taper part 143 in the direction L1.

The inclined surface of the under-head taper part 143 is formed in an annular shape around the axis L0 of the hook 23 (the central axis of the hook 23 which coincides with the axis which is the longitudinal axis of the front end sheath 15), and the cross-section thereof parallel to the axis L0 is formed in a linear shape. That is, the inclined surface of the under-head taper part 143 is inclined by an angle ψ from a plane perpendicular to the axis L0 and formed over the entire circumference. In this state, when a pulling force is applied to the manipulation wire, a resultant force vector W is produced at the abutting point O of the hook 23, in which the resultant force vector W acts as drag in relation to the front end 73a of the J-shaped claw part 73.

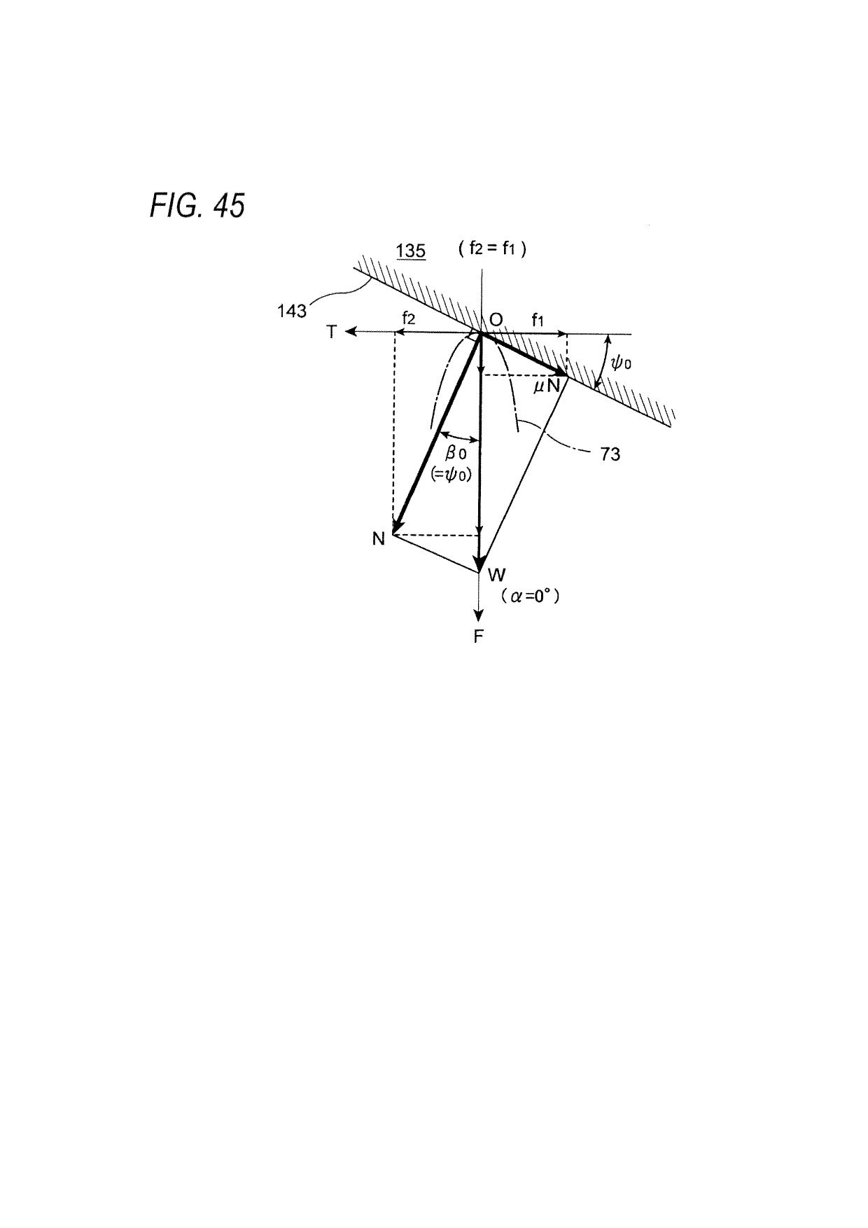

The resultant force vector W will be schematically described using FIG. 45.

Now, considered is a state in which the inclination angle of the under-head taper part 143 is set to ψ₀ so that the resultant force vector W will be produced in the direction parallel to the pulling direction by the manipulation wire, i.e., in the direction coinciding with the axis L0. The resultant force vector W may be decomposed into a normal force N which is perpendicular to the inclined surface of the under-head taper part 143, and a frictional force μN which is parallel to the inclined surface (μ is a frictional coefficient). The force T in the thrust direction, which is applied to the front end 73a of the J-shaped claw part 73 (see FIG. 44), i.e., to the point O, may be calculated by Equation (1) from the normal force N and the frictional force μN.

T=f₂−f₁=N sin ψ₀−μN cos ψ₀ (1)

Likewise, the force F in the direction of axis L0 may be calculated by Equation (2).

F=N cos ψ₀+N sin ψ₀ (2)

In addition, the force F may be expressed as Equation (3) from Equations (1) and (2).

F=T(sin ψ₀+μ cos ψ₀)/(cos ψ₀−μ sin ψ₀) (3)

When the thrust forces f₁ and f₂ by the resultant force vector W are equal to each other as illustrated in FIG. 45, the J-shaped claw part 73 will not move on the inclined surface of the under-head taper part 143 even if the pulling force from the manipulation wire is increased. In such a case, the engagement between the hook and the clip unit is not released.

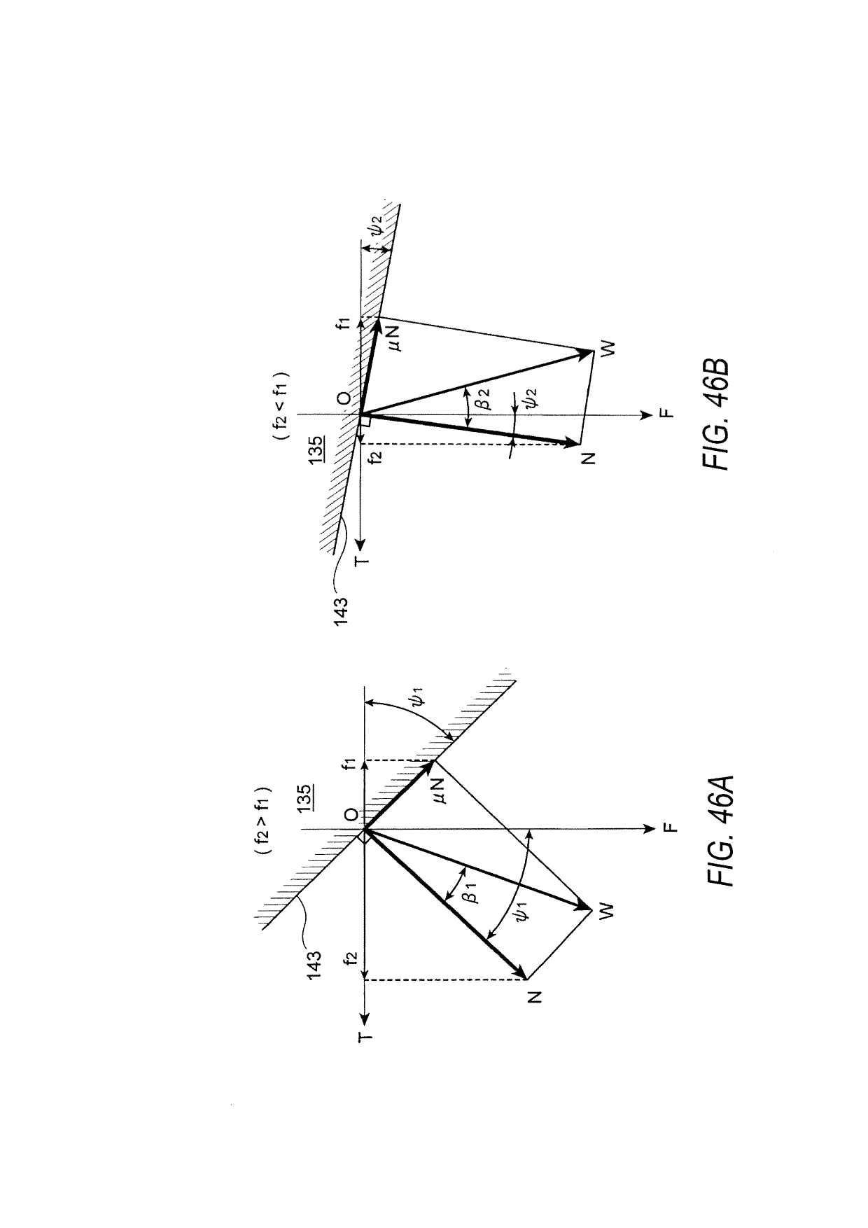

Meanwhile, as illustrated in FIG. 46A, if the inclination angle ψ₁ of the under-head taper part 143 is set to be larger than ψ₀ such that the thrust force f₂ is larger than the thrust force f₁, the J-shaped claw part 73 starts to be slid on the under-head taper part 143 to the left in the drawing. In such a case, the front end 73a of the J-shaped claw part 73 indicated at point O escapes from the outer periphery of the inclined surface of the under-head taper part 143 (see FIG. 38B), and finally, the engagement between the hook and the clip unit is released.

As illustrated in FIG. 46B, if the inclination angle ψ₂ is set to be smaller than ψ₀ such that the trust force f₁ is larger than f₂, the J-shaped claw part 73 starts to be slid on the inclined surface of the under-head taper part 143 to the right in the drawing. In such a case, the engagement between the chook and the clip unit is not released.

That is, in order to release the engagement between the hook and the clip unit, it is necessary to set the inclination angle ψ of the under-head taper part 143 to be larger than the inclination angle ψ₀ in the case where the resultant force vector W coincides with the pulling direction by the manipulation wire (in the axis L0).

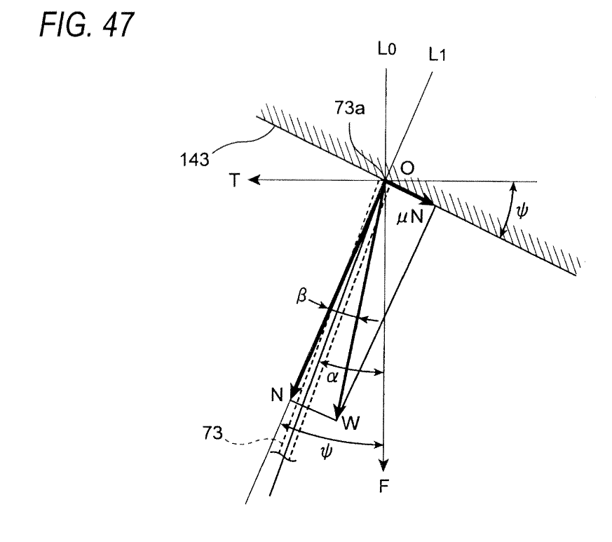

As to the abutting direction L1 of the J-shaped claw part 73 which abuts against the abutting point O of the under-head taper part 143 illustrated in FIG. 44, there also exists a requirement for releasing the engagement between the hook and the clip unit. As illustrated in FIG. 47, it is assumed that at the abutting point O where the inclined surface of the under-head taper part 143 abuts against the J-shaped claw part 73, the angle formed by the abutting direction L1 of the J-shaped claw part 73 and the axis L0 is α. It is also assumed that the angle formed by the resultant force vector W in relation to the direction normal to the inclined surface of the under-head taper part 143 at the abutting point O between the J-shaped claw part 73 and the inclined surface of the under-head taper part 143 is β, in which the resultant force vector W is produced at the abutting point by the pulling force from the manipulation wire.

At this time, as the abutting direction L of the J-shaped claw part 73 approaches the direction of the normal force N, the inclination of the under-head taper part 143 in relation to the front end 73a of the J-shaped claw part 73 at the abutting point O is substantially reduced, which makes it difficult for the J-shaped claw part 73 to be slid on the under-head taper part 143. That is, the angle of the abutting direction L which constitutes a limitation for causing the J-shaped claw part 73 to be slid on the under-head taper part 143 is an angle at which the direction of the resultant force vector W and the abutting direction L1 coincide with each other. In addition, when the inclination of the abutting direction of the front end 73a of the J-shaped claw part 73 is set to be ranged from an angle at which the inclination angle of the abutting direction from the axis L0 is smaller than that of the direction of the resultant force vector W or parallel to the axis L0 to an angle at which the front end of the extension direction of the J-shaped claw part 73 is directed radially outward in relation to the hook 23, the J-shaped claw part 73 starts to stably slide on the inclined surface of the under-head taper part 143.

From the above, when the relationship of the inclination angle ψ of the under-head taper part 143, the angle α and the angle β satisfy Equation (4), the J-shaped claw part 73 is slid on the under-head taper part 143, and the engagement between the hook and the clip unit is released.

α+β<ψ (4)

The conditions for satisfying Equation (4) do not include the state in which the inclination angle ψ of the under-head taper part 143 is not larger than ψ₀ like the states illustrated in FIGS. 45 and 46B.

FIG. 44 illustrates a state satisfying Equation (4), in which when the hook 23 is pulled back to the rear side (the lower side of the drawing), the front end 73a of the J-shaped claw part 73 is expanded diametrically outward while being slid. In addition, when the front end 73a of the J-shaped claw part 73 is expanded diametrically outward over the largest outer diameter of the hook part 135, the hook 23 escapes from the connection tail section 43, and hence the engagement between the hook 23 and the connection tail section 43 is released.

Like this, in the present construction, the resultant force for engagement release at the time of deforming the J-shaped claw part 73 of the connection tail section 43 to release the engagement can be correctly controlled by properly designing either the inclination angle of the under-head taper part 143 or the abutting direction of the J-shaped claw part 73. In addition, since the inclined surface of the under-head taper part 143 of the hook 23 has a linear shape when it is shown as a cross-section parallel to the axis L0, the sliding behavior of the J-shaped claw part 73 becomes uniform regardless of the position of the inclined surface, and the resultant force for engagement release becomes constant. Accordingly, it is possible to suppress the variation of the resultant force for engagement release. In addition, since the engagement and the engagement release are conducted at the same position, unnecessary fracture is not caused by the engagement release. For that reason, the process of loading the next clip can be continuously performed so that the length of time for maneuver can be reduced, and the construction of the ligation device can be simplified.

In addition, in accordance with the present construction, it is possible to control the to-be-deformed or to-be-fractured shape of the engagement claw, such as the J-shaped claw part 73, and to suppress the variation of the resultant force for release to the minimum. When the to-be-deformed or to-be-fractured shape of the engagement claw part is not controlled, considered are a shape configured to be bent by receiving an axial pressure, thereby being released the engagement, etc. beyond a shape configured to escape to the outside.

As described above, the inventive ligation device 100 is not limited to using it as a ligation device, and may also be converted into another treatment instrument for an endoscope that has a hook and an engagement claw part. In addition, it is possible to configure an endoscope system which enables a smooth endoscopic treatment by inserting the ligation device 100 through a treatment instrument inserting channel, through which a sheath member of an endoscope is inserted, and then introducing the ligation device 100 to the inside of a body cavity.

Like this, the present invention is not limited to the above-described exemplary embodiments. It is expected by the present invention that modifications and applications will be made by a person skilled in the art based on the disclosure of the specification and a well-known technology, and the modifications and applications are included in the scope to be protected.

As described above, the following items are disclosed in the present specification.

(1) A ligation device for ligating a biological tissue, includes:

a flexible elongated sheath member;

a manipulation unit arranged in a base end side of the sheath member;

a transmission member inserted into the sheath member to be capable of being reciprocated, and configured to transmit a driving force from the manipulation unit; and

a clip unit detachably attached to a front end of the transmission member opposite to the manipulation unit in a longitudinal axis of the sheath member;

in which the transmission member includes a hook fixed on a front end of the transmission member to be engaged with the clip unit, and

the hook includes a front side inclined surface and a rear side inclined surface formed in order from a front end side of the hook to a rear side thereof,

a sectional area of the front side inclined surface perpendicular to a central axis of the hook being gradually increased, and

a sectional area of the rear side inclined surface perpendicular to the central axis being gradually reduced,

in which the clip unit includes arms protruding from the rear end of the clip unit to define a space for accommodating the hook, and engagement claw parts respectively extending from rear ends of the arms toward a front side, and

in which, when the clip unit and the transmission member are connected, front ends of the engagement claw parts abut against the rear side inclined surface of the hook.

With this ligation device, when an action to extend the transmission member from the manipulation unit forward and to insert the hook into the clip unit positioned at the front end of the sheath member is performed, the hook is inserted into the engagement claw parts of the clip unit, and the engagement claw parts abut against the rear side inclined surface. In the state in which the hook is inserted to this position, when the transmission member is pulled backward, the engagement claw parts abut against the rear side inclined surface, thereby retaining the engagement, and when the pulling force is further increased, the engagement between the engagement claw parts and the rear side inclined surface is released. Due to this, the connection and connection release manipulations can be easily performed, and the resultant force for release at the time of performing the connection release can be more correctly set by the designed shape of the rear side inclined surface or the like, and the connection release can be stably performed.

(2) The ligation device according to (1), in which:

the clip unit includes a pair of arm parts, and a clip body having a connection base section connecting base ends of the arm parts; and

the arm extends from the connection base section in a direction opposite to the arm parts; and

the front ends of the engagement claw parts are located more rearward than the connection base section.

With this ligation device, since the front ends of the engagement claw parts are located more rearward than the connection base section of the clip body, the connection and connection release actions can be stably performed without being affected by the movement of the arm parts. In addition, since the engagement claw parts are formed integrally with the clip body having the arm parts, the connection and connection release actions can be performed with a simple construction without increasing the number of components.

(3) In the ligation device according to (2):

the front ends of the engagement claw parts are inclined toward the central axis of the hook more inwardly than the arm.

With this ligation device, since the front ends of the engagement claw parts are inclined more inwardly than the arm, the engagement claw parts can be stably engaged with the hook.

(4) In the ligation device according to any one of (1) to (3):

each of the engagement claw parts has a pair of claws which respectively extend toward the front side with reference to the rear end of the corresponding arm.

With this ligation device, since the pair of the engagement claw parts abut against the rear side inclined surface of the hook, it is possible to increase the connection strength between the clip unit and the hook.

(5) In the ligation device according to any one of (1) to (3):

each of the engagement claw parts is arranged in an opening formed in the corresponding arm, and provided in the opening to extend from the rear end of the arm toward the front side.

With this ligation device, since the engagement claw parts can be simply configured, it is easy to fabricate and assemble the ligation device.

(6) In the ligation device according to any one of (1) to (3):

the hook includes an expanded-diameter part on a rear side of the rear side inclined surface, the expanded-diameter part being capable of abutting against the engagement claw parts.

With this ligation device, since it is possible to obtain a centering effect as the expanded-diameter part abuts against the engagement claw parts, it is possible to engage the engagement claw parts and the front end inclined surface with each other more certainly.

(7) In the ligation device according to any one of (1) to (3):

the clip unit and the transmission member are connected by inserting the hook into the clip unit in the longitudinal axis of the sheath member.

With this ligation device, the connection manipulation of the clip unit and the hook can be simply performed.

(8) I the ligation device according to any one of (1) to (3):

when the hook is pulled back from the clip unit, the front ends of the engagement claw parts are expanded while being slid on the rear side inclined surface, and escape from the outer periphery of the rear side inclined surface, thereby releasing the connection.

With this ligation device, it is possible to set a resultant force for connection release between the hook and clip unit to an optional magnitude by controlling the inclination angle of the rear side inclined surface.

(9) In the ligation device according to any one of (1) to (3):

the cross-section of the rear side inclined surface parallel to the longitudinal axis of the sheath member is formed in a linear shape.

With this ligation device, since the rear side inclined surface has a linear shape, the engagement claw is adapted to be continuously slid with a predetermined and constant resultant force for connection release. Accordingly, the hook and the clip unit can be released from the connection by the predetermined and constant resultant force for connection release.

(10) In the ligation device according to any one of (1) to (3):

angles ψ, α, β satisfy the following relationship:

α+β<ψ

where ψ is an inclination angle between the rear side inclined surface and a plane perpendicular to the longitudinal axis of the sheath member when the engagement claw parts start to abut against the rear side inclined surface,

α is an angle formed between an abutting direction of one of the engagement claw parts at a abutting point of the rear side inclined surface with the corresponding engagement claw part, and the longitudinal axis of the sheath member, and

β is an angle formed by a resultant force vector produced at the abutting point of the engagement claw part and the rear side inclined surface by a pulling force from the transmission member, in relation to a direction perpendicular to the rear side inclined surface at the abutting point.

With this ligation device, when the relation of α+β<ψ is satisfied, the engagement claw parts are slid radially outward on the rear side inclined surface by the pulling force from the transmission member, thereby ensuring the connection release of the hook and the clip unit.

(11) In the ligation device according to any one of (1) to (3):

the clip unit is provided with a fastening ring which is arranged in an outside of the clip body to maintain the arm parts in a closed state.

With this ligation device, it is possible to stably maintain the ligation state in which the arm parts are closed by the fastening ring with a simple construction.

(12) A clip unit used in the ligation device according to any one of (1) to (11), includes:

the arm; and

the engagement claw parts.

With this clip unit, the manipulation of connection and connection release can be easily performed, the resultant force for release at the time of performing the connection release can be set to a predetermined constant resultant force, and the connection release can be stably performed.

(13) A clip manipulation device of the ligation device according to any one of (1) to (11), includes:

the sheath member;

the manipulation unit; and

the transmission member.

With this clip manipulation device, the manipulations of connection and connection release can be easily performed, the resultant force for release at the time of performing the connection release can be set to a predetermined constant resultant force, and the connection-releasing can be stably performed.

(14) An endoscope system includes:

the ligation device according to any one of (1) to (11); and

an endoscope including a treatment instrument insertion channel, through which the sheath member is inserted.

With this endoscope system, a smooth endoscope treatment can be performed.

What is claimed is:

1. A ligation device for ligating a biological tissue, comprising:
a flexible elongated sheath member;
a manipulation unit arranged in a base end side of the sheath member;
a transmission member inserted into the sheath member to be capable of being reciprocated, and configured to transmit a driving force from the manipulation unit; and
a clip unit detachably attached to a front end of the transmission member opposite to the manipulation unit in a longitudinal axis of the sheath member,
wherein the transmission member includes a hook fixed on a front end of the transmission member to be engaged with the clip unit, and
the hook includes a front side inclined surface and a rear side inclined surface formed in order from a front end side of the hook to a rear side thereof,
a sectional area of the front side inclined surface perpendicular to a central axis of the hook being gradually increased, and
a sectional area of the rear side inclined surface perpendicular to the central axis being gradually reduced,
wherein the clip unit includes arms protruding from the rear end of the clip unit to define a space for accommodating the hook, and engagement claw parts respectively extending from rear ends of the arms toward a front side, and
wherein, when the clip unit and the transmission member are connected, front ends of the engagement claw parts abut against the rear side inclined surface of the hook.

2. The ligation device according to claim 1, wherein:
the clip unit includes a pair of arm parts, and a clip body having a connection base section connecting base ends of the arm parts;
the arm extends from the connection base section in a direction opposite to the arm parts; and
the front ends of the engagement claw parts are located more rearward than the connection base section.

3. The ligation device according to claim 2, wherein:
the front ends of the engagement claw parts are inclined toward the central axis of the hook more inwardly than the arm.

4. The ligation device according to claim 1, wherein:
each of the engagement claw parts has a pair of claws which respectively extend toward the front side with reference to the rear end of the corresponding arm.

5. The ligation device according to claim 1, wherein:
each of the engagement claw parts is arranged in an opening formed in the corresponding arm, and provided in the opening to extend from the rear end of the arm toward the front side.

6. The ligation device according to claim 1, wherein:
the hook includes an expanded-diameter part on a rear side of the rear side inclined surface, the expanded-diameter part being capable of abutting against the engagement claw parts.

7. The ligation device according to claim 1, wherein:
the clip unit and the transmission member are connected by inserting the hook into the clip unit in the longitudinal axis of the sheath member.

8. The ligation device according to claim 1, wherein:
when the hook is pulled back from the clip unit, the front ends of the engagement claw parts are expanded while being slid on the rear side inclined surface, and escape from the outer periphery of the rear side inclined surface, thereby releasing the connection.

9. The ligation device according to claim 1, wherein:
the cross-section of the rear side inclined surface parallel to the longitudinal axis of the sheath member is formed in a linear shape.

10. The ligation device according to claim 1, wherein:
angles ψ, α, β satisfy the following relationship:
α+β<ψ
where ψ is an inclination angle between the rear side inclined surface and a plane perpendicular to the longitudinal axis of the sheath member when the engagement claw parts start to abut against the rear side inclined surface,
α is an angle formed between an abutting direction of one of the engagement claw parts at a abutting point of the rear side inclined surface with the corresponding engagement claw part, and the longitudinal axis of the sheath member, and
β is an angle formed by a resultant force vector produced at the abutting point of the engagement claw part and the rear side inclined surface by a pulling force from the transmission member, in relation to a direction perpendicular to the rear side inclined surface at the abutting point.

11. The ligation device according to claim 1, wherein:
the clip unit is provided with a fastening ring which is arranged in an outside of the clip body to maintain the arm parts in a closed state.

12. A clip unit used in the ligation device according to claim 1, comprising:
the arm; and
the engagement claw parts.

13. A clip manipulation device of the ligation device according to claim 1, comprising:
the sheath member;
the manipulation unit; and
the transmission member.

14. An endoscope system comprising:
the ligation device according to claim 1; and
an endoscope including a treatment instrument insertion channel, through which the sheath member is inserted.
